SEQUENCE LISTING (6 sequences)

SEQ ID NO: 1 - DNA, 21 bp, Artificial Sequence
  Forward primer IL-6 mouse gene
gaacaacgatgatgcacttgc

SEQ ID NO: 2 - DNA, 21 bp, Artificial Sequence
  Reverse primer IL-6 mouse gene
tccaggtagctatggtactcc

SEQ ID NO: 3 - DNA, 20 bp, Artificial Sequence
  Forward primer TNFalpha mouse gene
agaggcactcccccaaaaga

SEQ ID NO: 4 - DNA, 22 bp, Artificial Sequence
  Reverse primer TNFalpha mouse gene
cgatcaccccgaagttcccatt

SEQ ID NO: 5 - DNA, 22 bp, Artificial Sequence
  Forward primer GAPDH mouse gene
tggcaaagtggagattgttgcc

SEQ ID NO: 6 - DNA, 21 bp, Artificial Sequence
  Reverse primer GAPDH mouse gene
aagatggtgatgggcttcccg

(19) United States
(12) Patent Application Publication
(10) Pub. No.: US 2022/0041538 A1
(43) Pub. Date: Feb. 10, 2022

(54) CANNABIGEROL QUINONE ACID AND SALTS THEREOF

(71) Applicant: EMERALD HEALTH PHARMACEUTICALS INC, San Diego, CA (US)

(72) Inventors: Eduardo MUÑOZ BLANCO, Cordoba (ES); Giovanni APPENDINO, Torino (IT)

(73) Assignee: EMERALD HEALTH PHARMACEUTICALS INC, San Diego, CA (US)

(21) Appl. No.: 17/413,199

(22) PCT Filed: Dec. 11, 2019
(86) PCT No.: PCT/EP2019/084764

§ 371 Date: Jun. 11, 2021

(30) Foreign Application Priority Data
Dec. 11, 2018 (EP) 18382908.4

(51) Int. Cl. C07C 66/00

(52) U.S. Cl. CPC C07C 66/00

(57) ABSTRACT

A compound of formula I

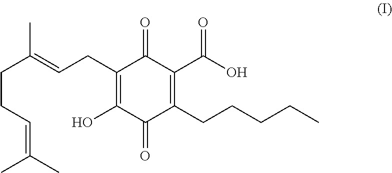 [SMILES: CCCCCC1=C(C(=O)O)C(=O)C(C/C=C(\C)CCC=C(C)C)=C(O)C1=O]

or a pharmaceutical salt thereof of formula II,

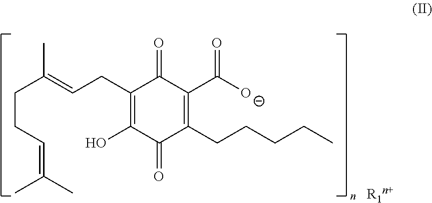

as well as a process to obtain said compound and a process to obtain said salt. Additionally, disclosed is the use of said compound of formula I or said pharmaceutical salt thereof of formula II as a medicament, in particular as a peroxisome proliferator-activated receptor gamma (PPARγ) agonist, for use in the treatment or prevention of a disease responsive to PPARγ agonists. Also disclosed is a pharmaceutical composition comprising said compound or said salt, as well as a method of treating or preventing a disease with said compound of formula I or said salt thereof of formula II, or with a composition comprising said compound or said salt.

### FIELD OF THE INVENTION

The present invention relates to cannabigerol quinone acid and salts thereof, as well as the synthesis of said acid and salts thereof. Additionally, the present invention relates to the use of said cannabigerol quinone acid and salts thereof.

### BACKGROUND OF THE INVENTION

Nuclear receptors (NRs) are a major target of drug discovery. NRs are ligand-dependent transcription factors that possess the ability to directly interact with DNA regulating the transcriptional activity of their target genes. These receptors play essential roles in development, cellular homeostasis and metabolism.

In the nomenclature for nuclear receptors, the peroxisome proliferator-activated receptor (PPAR) group of the nuclear subfamily 1 C (NR1C) comprises three subtypes of mammalian PPARs: PPARα (also called NR1C1), PPARβ/δ (also called NR1C2) and PPARγ (also called PPARγ, glitazone receptor or NR1C3).

Quinones represent a class of toxicological intermediates, which can create a variety of hazardous effects in vivo, including acute cytotoxicity and immunotoxicity. The mechanisms by which quinones cause these effects can be quite complex. Quinones are Michael acceptors, and cellular damage can occur through alkylation of crucial cellular proteins and/or DNA. Alternatively, quinones are highly redox active molecules which can redox cycle with their semiquinone radicals, leading to formation of reactive oxygen species (ROS) that can cause severe oxidative stress within cells through the formation of oxidized cellular macromolecules, including lipids, proteins, and DNA. Although there are numerous examples of quinone-based compounds with therapeutic use, due to the concerns over non-specific toxicity and lack of selectivity, the Michael acceptor motif is rarely introduced by design in drug leads.

One example of quinone-based therapeutic compounds is reported in WO2011/117429, wherein the synthesis of cannabigerol hydroxy-quinone (also named CBG-Q or VCE-003 in the aforesaid international patent application) is described, together with its use in diseases and conditions responsive to PPARγ modulation. Diseases responsive to PPARγ modulation are, as included in WO2011/117429: atherosclerosis, inflammatory bowel diseases, rheumatoid arthritis, liver fibrosis, nephropathy, psoriasis, skin wound healing, skin regeneration, pancreatitis, gastritis, neurodegenerative disorders, cancer; hypertension, hypertriglyceridemia, hypercholesterolemia, obesity and type II diabetes. The introduction of a quinone motif in the cannabigerol molecule increases its affinity to PPARγ and increases its transcriptional activity.

Further research shows that cannabigerol hydroxy-quinone (CBG-Q), described in WO2011/117429, also activates the transcription factor Nrf2, a cellular sensor of oxidative/electrophilic stress. Thus, introduction of a quinone motif in cannabigerol results in two independent activities such as those exerted as PPARγ agonists and Nrf2 activators. WO2015/128200 discloses compounds suitable for treating PPARγ-related diseases which, due to specific modifications in position 2, exhibit PPARγ agonistic effects but lack electrophilic (Nrf2 activation) and cytotoxic activities.

Thus, there is the need to provide compounds that exhibit a PPARγ agonistic effect but lack electrophilic (Nrf2 activation) and cytotoxic activity which, to date, have not been possible to synthesize. Said compounds necessarily should be suitable for pharmaceutical use in the treatment or prevention of diseases and conditions responsive to PPARγ modulation. Preferably, said compounds exhibit improved pharmacodynamic and pharmacokinetic properties over compounds that exhibit a PPARγ agonistic effect but lack electrophilic (Nrf2 activation) and cytotoxic activity which are described in the prior art.

### BRIEF DESCRIPTION OF THE INVENTION

The present invention relates to a compound of formula I:

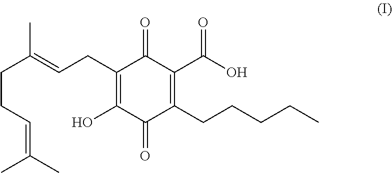 [SMILES: CCCCCC1=C(C(=O)O)C(=O)C(C/C=C(\C)CCC=C(C)C)=C(O)C1=O]

or to a pharmaceutical salt of formula I. In one embodiment the pharmaceutical salt of formula I is represented by formula II:

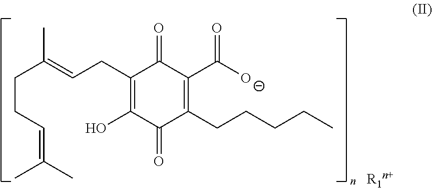

wherein R₁ⁿ⁺ is selected from the group consisting of:

a metal cation;

an amino acid cation; and

an ammonium cation of formula III:

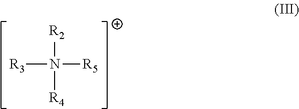

wherein R₂, R₃, R₄ and R₅ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or two of R₂, R₃, R₄ and R₅ are linked to form a heterocyclic group; and

a guanidinium cation of formula (IV):

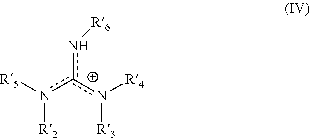

wherein R′₂, R′₃, R′₄, R′₅ and R′₆ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or wherein two of R′₂, R′₃, R′₄, R′₅ and R′₆ are linked to form a heterocyclic group,

wherein n is a number selected from the group consisting of: 1, 2, 3 and 4.

The present invention also refers to a pharmaceutical salt of formula II:

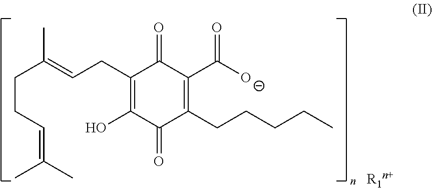

wherein R₁ⁿ⁺ is selected from the group consisting of:
an alkali metal cation or an alkaline earth metal cation;
an amino acid cation; and
an ammonium cation of formula III:

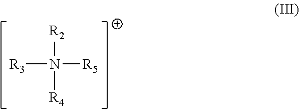

wherein R₂, R₃, R₄ and R₅ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or two of R₂, R₃, R₄ and R₅ are linked to form a heterocyclic group,

wherein n is a number selected from 1 or 2.

The present invention also relates to a pharmaceutical salt of formula II:

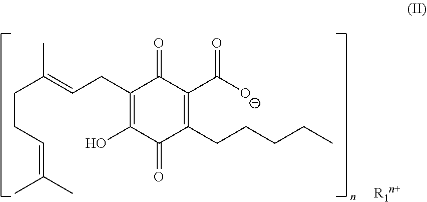

wherein the pharmaceutical salt of formula II is a salt comprising an anion of a compound of formula I:

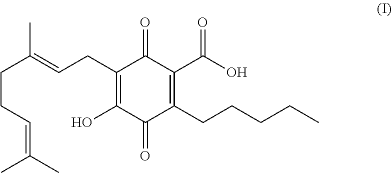 [SMILES: CCCCCC1=C(C(=O)O)C(=O)C(C/C=C(\C)CCC=C(C)C)=C(O)C1=O]

and a cation selected from the group consisting of:

a cation of an alkali metal base,

a cation of an alkaline earth metal base, and

a cation derived, by protonation, from a compound selected from the group consisting of: L-lysine, L-arginine, trimethylamine, propylamine, methylamine, isopropylamine, butylamine, diethylamine, 2-(dimethylamine) ethanol, tromethamine, meglumine, cyclobutylamine, cyclopropanemethylamine, dicyclohexylamine, 1-bicyclo[1.1.1]pentylamine, ethylendiamine, diaminopropane, aniline, pyridine, quinoline, phenylenediamine and benzathine,

wherein n is a number selected from 1 or 2.

In addition, the present invention also relates to a process to obtain a compound of formula I:

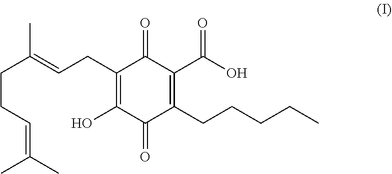 [SMILES: CCCCCC1=C(C(=O)O)C(=O)C(C/C=C(\C)CCC=C(C)C)=C(O)C1=O]

wherein said process comprises the steps of
a. oxidizing cannabigerolic acid (CBGA) with an oxidizing agent in an aprotic solvent, in the presence of a base having a pKa of at least 11.5, wherein said pKa is measured in water at 25° C., to obtain a compound of formula I:

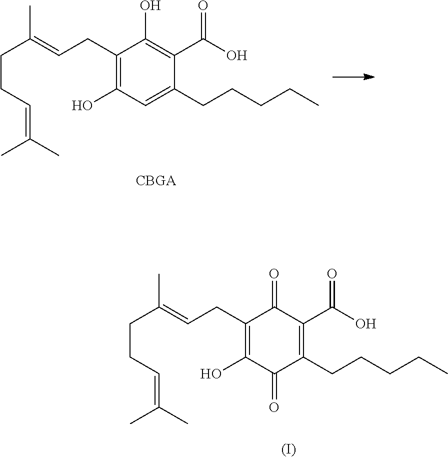

b. isolating the compound of formula I.

The present invention also relates to a process to obtain a compound of formula I:

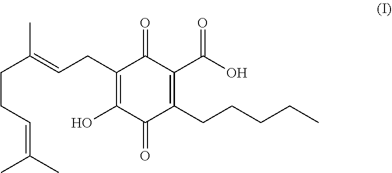 [SMILES: CCCCCC1=C(C(=O)O)C(=O)C(C/C=C(\C)CCC=C(C)C)=C(O)C1=O]

wherein said process comprises the steps of:
a. oxidizing cannabigerolic acid (CBGA) with an oxidizing agent in an aprotic solvent, in the presence of a base having a pKa of at least 11.5, wherein said pKa is measured in water at 25° C., to obtain a compound of formula I:

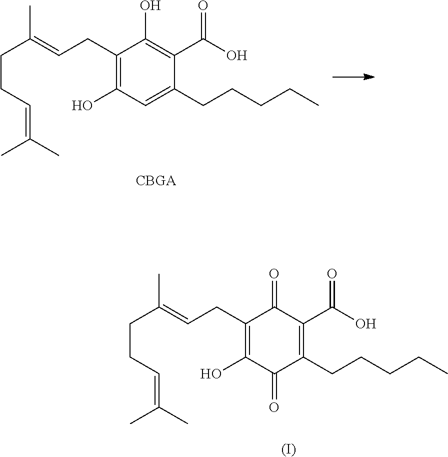

b. isolating the compound of formula I,

wherein: said oxidizing agent is air; said base is selected from an alkali metal alkoxide, an alkaline earth metal alkoxide or an alkali metal alkylsilylamine; and said aprotic solvent is selected from the group consisting of toluene, tetrahydrofuran, 1,4-dioxane, 2-methyltetrahydrofuran and ethyl acetate.

Additionally, the present invention also relates to a process to obtain a pharmaceutical salt of formula II:

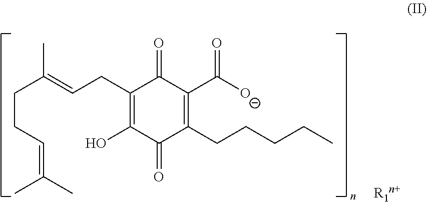

wherein R₁ⁿ⁺ is:
a metal cation; an amino acid cation; or an ammonium cation of formula III:

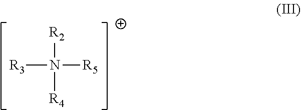

wherein R₂, R₃, R₄ and R₅ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or two of R₂, R₃, R₄ and R₅ are linked to form a heterocyclic group; and
a guanidinium cation of formula (IV):

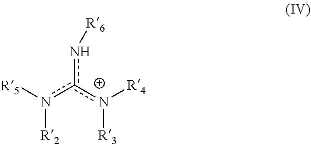

wherein R′₂, R′₃, R′₄, R′₅ and R′₆ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or wherein two of R′₂, R′₃, R′₄, R′₅ and R′₆ are linked to form a heterocyclic group; and

wherein said process comprises:

i. when R₁ⁿ⁺ is a metal cation:
i.a. contacting a solution of the compound of formula I with said metal cation; or
i.b. contacting a solution of the compound of formula I with a first cation to form a salt of the compound of formula I and said first cation; and contacting said salt of the compound of formula I and said first cation with said metal cation; or
i.c. contacting a solution of the compound of formula I with the metal from which said metal cation is derived or an inorganic compound of said metal;

ii. when R₁ⁿ⁺ is an amino acid cation:
ii.a contacting a solution of the compound of formula I with said amino acid cation; or
ii.b. contacting a solution of the compound of formula I with a first cation to form a salt of the compound of formula I and said first cation; and contacting said salt of the compound of formula I and said first cation with said amino acid cation; or
ii.c contacting a solution of the compound of formula I with the amino acid from which said amino acid cation is derived by protonation;
iii. when R₁ⁿ⁺ is an ammonium cation of formula III:
iii.a. contacting a solution of the compound of formula I with said ammonium cation of formula III; or
iii.b contacting as solution of the compound of formula I with a first cation to form a salt of the compound of formula I and said first cation; and contacting said salt of the compound of formula I and said first cation with said ammonium cation of formula III; or
iii.c. and when R₅ is H, contacting a solution of the compound of formula I with the amine of formula V from which said ammonium cation of formula III is derived by protonation:

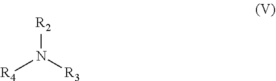

iv. when R₁ⁿ⁺ is a guanidinium cation of formula IV:
iv.a. contacting the compound of formula I with said guanidinium cation of formula IV; or
iv.b. contacting as solution of the compound of formula I with a first cation to form a salt of the compound of formula I and said first cation; and contacting said salt of the compound of formula I and said first cation with said guanidinium cation of formula IV; or
iv.c. contacting a solution of the compound of formula I with a guanidine derivative of formula IVb from which said guanidium cation of formula IV is derived by protonation:

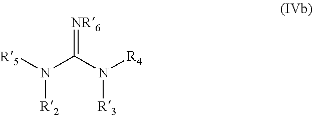

wherein n is a number selected from the group consisting of: 1, 2, 3 and 4.

Further, the present invention also relates to a process to obtain a pharmaceutical salt of formula II:

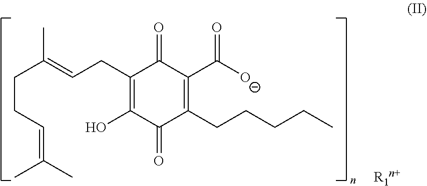

wherein R₁ⁿ⁺ is:
a metal cation;
an amino acid cation; or
an ammonium cation of formula III:

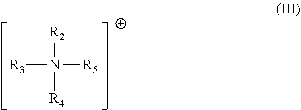

wherein R₂, R₃, R₄ and R₅ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or two of R₂, R₃, R₄ and R₅ are linked to form a heterocyclic group; and
a guanidinium cation of formula (IV):

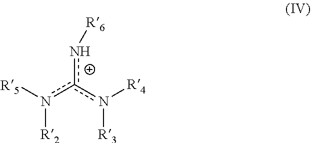

wherein R′₂, R′₃, R′₄, R′₅ and R′₆ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or wherein two of R′₂, R′₃, R′₄, R′₅ and R′₆ are linked to form a heterocyclic group; and

wherein said process comprises:
i.a. when R₁ⁿ⁺ is a metal cation, contacting a solution of the compound of formula I compound of formula I with the metal cation;
ii.c. when R₁ⁿ⁺ is an amino acid cation, contacting a solution of the compound of formula I with the amino acid from which said amino acid cation is derived by protonation;

iii. when R₁ⁿ⁺ is an ammonium cation of formula III:
iii.a. contacting a solution of the compound of formula I with said ammonium cation of formula III; or
iii.b contacting as solution of the compound of formula I with a first cation to form a salt of the compound of formula I and said first cation; and contacting said salt of the compound of formula I and said first cation with said ammonium cation of formula III; or
iii.c. and when R₅ is H, contacting a solution of the compound of formula I with the amine of formula V from which said ammonium cation of formula III is derived by protonation:

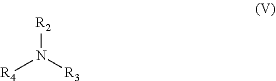

iv.c. when R₁ⁿ⁺ is a guanidinium cation of formula IV, contacting a solution of the compound of formula I with the guanidine derivative of formula IVb from which said guanidium cation of formula IV is derived by protonation

preferably wherein:

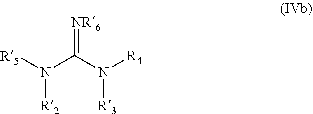

said metal cation is an alkali metal cation or an alkaline earth metal cation,
said amino acid cation is a cation derived from L-lysine or L-arginine by protonation, and
said ammonium cation of formula III is a cation derived from trimethylamine, propylamine, methylamine, isopropylamine, butylamine, diethylamine, 2-(dimethylamino)-ethanol, tromethamine, meglumine, cyclobutylamine, cyclopropanemethylamine, dicyclohexylamine, 1-bicyclo[1.1.1]pentylamine, ethylendiamine, diaminopropane, aniline, pyridine, quinoline, phenylenediamine or benzathine by protonation,

and wherein n is a number selected from the group consisting of: 1, 2, 3 and 4.

Furthermore, the present invention relates to a compound of formula I, or a pharmaceutical salt thereof of formula II, according to present invention, for use as a medicament.

Moreover, the present invention relates to a compound of formula I, or a pharmaceutical salt thereof of formula II, according to present invention, for use in the treatment or prevention of a disease responsive to PPARγ agonists.

The present invention also relates to a pharmaceutical salt of formula II:

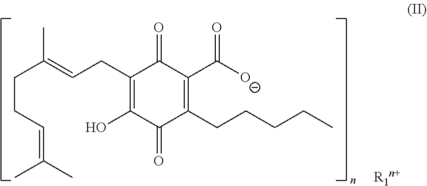

wherein the pharmaceutical salt of formula II is a salt comprising an anion of the compound of formula I:

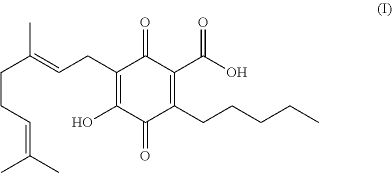 [SMILES: CCCCCC1=C(C(=O)O)C(=O)C(C/C=C(\C)CCC=C(C)C)=C(O)C1=O]

and a cation selected from the group consisting of:

a cation derived from an alkali metal inorganic compound,

a cation derived from an alkaline earth metal inorganic compound, and

a cation derived, by protonation, from a compound selected from the group consisting of: L-lysine, L-arginine, trimethylamine, propylamine, methylamine, isopropylamine, butylamine, diethylamine, 2-(dimethylamino)-ethanol, tromethamine, meglumine, cyclobutylamine, cyclopropanemethylamine, dicyclohexylamine, 1-bicyclo[1.1.1]pentylamine, ethylendiamine, diaminopropane, aniline, pyridine, quinoline, phenylenediamine and benzathine;

for use in the treatment or prevention of a disease responsive to PPARγ agonists, wherein the disease responsive to PPARγ agonists is selected from the group consisting of atherosclerosis, inflammatory bowel diseases, rheumatoid arthritis, liver fibrosis, nephropathy, psoriasis, skin wound healing, skin regeneration, pancreatitis, gastritis, neurodegenerative disorders, neuroinflammatory disorders, scleroderma, cancer, hypertension, obesity and type II diabetes; wherein n is a number selected from 1 or 2.

In some aspects, the present invention relates to methods for treating or preventing a disease responsive to PPARγ agonists comprising administering to a patient an effective amount of a compound of formula I or a pharmaceutical salt thereof of formula II according to present invention.

### BRIEF DESCRIPTION OF THE FIGURES

FIG. 1: Relative affinity for PPARγ represented as percentage of polarization plotted against the concentration (as Log of concentration) of the tromethamine salt (IIa) of the cannabigerol quinone acid of formula II.

FIG. 2: Relative affinity for PPARγ represented as percentage of polarization plotted against the concentration (as Log of concentration) of the ethylenediamine salt (IIb) of the cannabigerol quinone acid of formula II.

FIG. 3: Relative affinity for PPARγ represented as percentage of polarization plotted against the concentration (as Log of concentration) of the benzathine salt (IIc) of the cannabigerol quinone acid of formula II.

FIG. 4: Relative affinity for PPARγ represented as percentage of polarization plotted against the concentration (as Log of concentration) of the calcium salt (IId) of the cannabigerol quinone acid of formula II.

FIG. 5: Relative affinity for PPARγ represented as percentage of polarization plotted against the concentration (as Log of concentration) of the sodium salt (IIe) of the cannabigerol quinone acid of formula II.

FIG. 6: Relative affinity for PPARγ represented as percentage of polarization plotted against the concentration (as Log of concentration) of the dicyclohexylamine salt (IIf) of the cannabigerol quinone acid of formula II.

FIG. 7: Relative affinity for PPARγ represented as percentage of polarization plotted against the concentration (as Log of concentration) of the L-arginine salt (IIg) of the cannabigerol quinone acid of formula II.

FIG. 8: Relative affinity for PPARγ represented as percentage of polarization plotted against the concentration (as Log of concentration) of the meglumine salt (IIh) of the cannabigerol quinone acid of formula II.

FIG. 9: Relative affinity for PPARγ represented as percentage of polarization plotted against the concentration (as Log of concentration) of the L-lysine salt (IIi) of the cannabigerol quinone acid of formula II.

FIG. 10: Relative affinity for PPARγ represented as percentage of polarization plotted against the concentration (as Log of concentration) of the potassium salt (IIj) of the cannabigerol quinone acid of formula II.

FIG. 11: Relative affinity for PPARγ represented as percentage of polarization plotted against the concentration (as Log of concentration) of the 2-dimethylamino-ethanol salt (IIk) of the cannabigerol quinone acid of formula II.

FIG. 12: Relative affinity for PPARγ represented as percentage of polarization plotted against the concentration (as Log of concentration) of the cannabigerol quinone acid (I).

FIG. 13: PPARγ transactivation assays in 293T cells. The concentration of the tested compounds (μM) is shown at the x-axis and the PPARγ inductions fold is shown at the y-axis. VCE-003 was used as comparative control. Fold activation level was calculated, taking the control sample (-), without the presence of any PPARγ agonist or activating agent, as reference. Data are expressed as mean±S.D. of at least three independent experiments.

FIG. 14: Behavioral score in mice after 3NP (3-nitropropionic acid) intoxication and after treatment with cannabigerol quinone acid (I).

Mice were subjected to behavioral tests for determining their neurological status after the treatment with oral cannabigerol quinone acid (I) (10 mg/Kg dissolved in sesame oil) and after intraperitoneal delivery (10 mg/Kg dissolved in ethanol/cremophor/saline). Hind limb clasping, Locomotor activity, and kyphosis were rated from 0 to 2 based on severity: a score of 0 typically indicates normal function and 2 seriously affected. Values are expressed as means±SEM for 6 animals per group.

FIG. 15. Behavioral score in mice after 3NP intoxication and after treatment with sodium salt (IIe) of the cannabigerol quinone acid of formula II.

Mice were subjected to behavioral tests for determining their neurological status after the treatment with oral sodium salt of the cannabigerol quinone acid (30 mg/Kg dissolved saline) and after intraperitoneal delivery (10 mg/Kg dissolved in saline). Hind limb clasping, Locomotor activity, and kyphosis were rated from 0 to 2 based on severity: a score of 0 typically indicates normal function and 2 seriously affected. Values are expressed as means±SEM for 6 animals per group.

FIG. 16. Neuroprotective and anti-inflammatory activity of cannabigerol quinone acid (I) in 3NP-intoxicated mice.

Loss of neurons in the striatum (Nissl staining) and Iba1 (microglia marker) were detected by immunostaining in the coronal sections of striatum of mice treated with vehicle, 3NP+ vehicle, 3NP+ compound I (oral and intraperitoneal). Quantification of Nissl staining (A) and Iba1 (B) positive cells in the mouse striatum. Total average number of neurons and microglia is shown. Values are expressed as means±SEM for 3 animals per group.

FIG. 17. Neuroprotective and anti-inflammatory activity of the sodium salt (IIe) of the cannabigerol quinone acid of formula II in 3NP-intoxicated mice.

Loss of neurons in the striatum (Nissl staining) and Iba1 (microglia marker) were detected by immunostaining in the coronal sections of striatum of mice treated with vehicle, 3NP+ vehicle, 3NP+ sodium salt of the cannabigerol quinone acid of formula II (oral and intraperitoneal). Quantification of Nissl staining (A) and Iba1 (B) positive cells in the mouse striatum. Total average number of neurons and microglia is shown. Values are expressed as means±SEM for 3 animals per group.

FIG. 18. Effect of Cannabigerol quinone acid (I) on the expression of proinflammatory mediators in the brain of 3NP-intoxicated mice.

Gene expression of inflammatory markers TNFα (A) and IL-6 (B) was down regulated in 3NP+ cannabigerol quinone acid (I)-treated mice (10 mg/kg, oral and intraperitoneal) compared with 3NP+Vehicle mice. Expression levels were calculated using the 2^{-ΔΔCt} method. Values are expressed as means±SEM for 3 animals per group.

FIG. 19. Effect of the sodium salt (IIe) of the cannabigerol quinone acid of formula II on the expression of proinflammatory mediators in the brain of 3NP-intoxicated mice.

Gene expression of inflammatory markers TNFα (A) and IL-6 (B) was down regulated in 3NP+ sodium salt (IIe) of the cannabigerol quinone acid of formula II treated mice (30 mg/kg, oral and 10 mg/Kg intraperitoneal) compared with 3NP+Vehicle mice. Expression levels were calculated using the 2^{-ΔΔCt} method. Values are expressed as means±SEM for 3 animals per group.

FIG. 20. Cannabigerol quinone acid (I) alleviates clinical symptoms in 6-hydroxy dopamine (6-OH-DA) challenged mice.

C57BL/6 mice were unilaterally injected intracerebroventricullarly with 6-hydroxydopamine (6-OHDA) or saline (control mice) and subjected to chronic intraperitoneal treatment with cannabigerol quinone acid (1) (oral 20 mg/mL in sesame oil, and intraperitoneal 10 mg/Kg in Tween80/Saline ( 1/16)) or vehicle (14 days), starting 16 h after the 6-OHDA injection. SHAM group corresponds to mice subjected to the surgical manipulation without injection of 6-OHDA. Motor coordination was evaluated by rotarod performance and motor activity was evaluated using a computer-aided actimeter. A: pole test results after oral treatment, B: cylinder rearing test results after oral treatment, C: pole test results after intraperitoneal treatment, D: cylinder rearing test results after intraperitoneal treatment. Values are expressed as means±SEM for 6 animals per group.

FIG. 21. Sodium salt of the cannabigerol quinone acid of formula II (compound lie) alleviates clinical symptoms in 6-OH-DA challenged mice.

C57BL/6 mice were unilaterally injected intracerebroventricullarly with 6-hydroxydopamine (6-OHDA) or saline (control mice) and subjected to chronic intraperitoneal treatment with sodium salt of the cannabigerol quinone acid of formula II dissolved in saline (oral 40 mg/mL and intraperitoneal 10 mg/kg) or vehicle (14 days), starting 16 hours after the 6-OHDA injection. Motor coordination was evaluated by rotarod performance and motor activity was evaluated using a computer-aided actimeter. A: pole test results after oral treatment, B: cylinder rearing test results after oral treatment, C: pole test results after intraperitoneal treatment, D: cylinder rearing test results after intraperitoneal treatment. Values are expressed as means±SEM for 6 animals per group.

### DESCRIPTION OF THE INVENTION

Present invention refers to a compound of formula I:

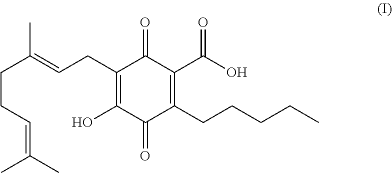 [SMILES: CCCCCC1=C(C(=O)O)C(=O)C(C/C=C(\C)CCC=C(C)C)=C(O)C1=O]

or to a pharmaceutical salt of formula II of said compound of formula I:

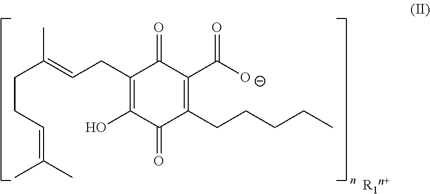

wherein R₁ⁿ⁺ is selected from the group consisting of:
a metal cation;
an amino acid cation; and
an ammonium cation of formula III:

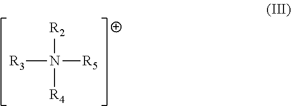

wherein R₂, R₃, R₄ and R₅ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or two of R₂, R₃, R₄ and R₅ are linked to form a heterocyclic group; and
a guanidinium cation of formula (IV):

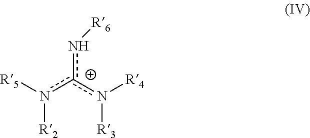

wherein R′₂, R′₃, R′₄, R′₅ and R′₆ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or wherein two of R′₂, R′₃, R′₄, R′₅ and R′₆ are linked to form a heterocyclic group; and

wherein n is a number selected from the group consisting of: 1, 2, 3 and 4.

The present invention also relates to a pharmaceutical composition comprising a compound of said formula I or said pharmaceutical salt thereof of formula II. The present invention also refers to said compound of formula I or said pharmaceutical salt thereof of formula II for use as a medicament, as well as to said compound of formula I or said pharmaceutical salt thereof of formula II for use in the treatment or prevention of a disease responsive to PPARγ agonists. Analogously, the present invention also refers to a method for treating or preventing a disease responsive to PPARγ agonists comprising administering to a patient an effective amount of said compound of formula I or said pharmaceutical salt thereof of formula II. The present invention also refers to a process to obtain said compound of formula I and a process to obtain said pharmaceutical salt of formula II.

The compound of formula I, and pharmaceutical salts of formula II thereof described herein, also comprise their tautomeric forms, isomers, stereoisomers, polymorphs, and compositions containing the same.

One embodiment of the present invention relates to a compound of formula (I):

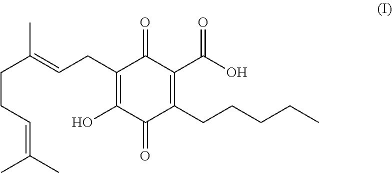 [SMILES: CCCCCC1=C(C(=O)O)C(=O)C(C/C=C(\C)CCC=C(C)C)=C(O)C1=O]

Another embodiment of the present invention relates to a pharmaceutical salt of the compound of formula I, of formula (II):

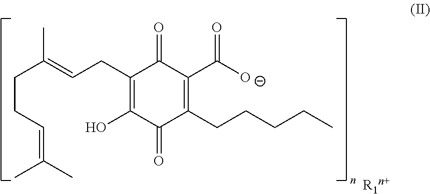

wherein n is a number selected from the group consisting of: 1, 2, 3 and 4.

The index “n” refers to the charge of the cation R₁ⁿ⁺ and also to the number of carboxylate anions in formula II, and is a whole number selected from the group consisting of: 1, 2, 3 and 4, preferably 1 or 2.

In a preferred embodiment, R₁ⁿ⁺ is a metal cation wherein said metal cation is an alkali metal cation or an alkaline earth metal cation, more preferably R₁ⁿ⁺ is Ca²⁺ (whereby n is 2), or K⁺ or Na⁺ (whereby n is 1).

In a preferred embodiment, R₁ⁿ⁺ is an ammonium cation of formula III, wherein R₂, R₃, R₄ and R₅ are each independently selected from the group consisting of: H, alkyl, hydroxyalkyl, poly(hydroxy)alkyl, cycloalkyl, arylalkyl, aminoalkyl and arylalkylaminoalkyl. In a more preferred embodiment alkyl is C₁₋₆ alkyl, most preferably selected from the group consisting of methyl, ethyl, propyl and butyl. In a preferred embodiment, hydroxyalkyl is C₁₋₆ hydroxyalkyl, more preferably selected from the group consisting of hydroxymethyl, hydroxyethyl, 1-hydroxypropyl, 2-hydroxypropyl and hydroxybutyl. In a preferred embodiment poly(hydroxy)alkyl is selected from the group consisting of ethanyl-1,2-diol, 2-(hydroxymethyl)propanyl-1,3-diol, butanyl-1,2,3,4-tetrol, (2R,4R)-pentanyl-1,2,3,4,5-pentol, (2R,3R,4R,5S)-hexanyl-1,2,3,4,5-pentol and (2R,3R,4R,5R)-hexanyl-1,2,3,4,5,6-hexol. In a preferred embodiment cycloalkyl is selected from the group consisting of cyclopropyl, cyclobutyl, cyclohexyl and bicyclohexyl. In a more preferred embodiment arylalkyl is benzyl. In a preferred embodiment aminoalkyl is C1-6 aminoalkyl, most preferably selected from the group consisting of aminomethyl and aminoethyl. In a preferred embodiment arylalkylaminoalkyl is benzylaminoethyl.

In a preferred embodiment R₁ⁿ⁺ is selected from the group consisting of an alkali metal cation, an alkaline earth metal cation, and an ammonium cation of formula III, wherein R₂, R₃, R₄ and R₅ are each independently selected from the group consisting of: H, methyl, ethyl, propyl, butyl, hydroxymethyl, hydroxyethyl, 1-hydroxypropyl, 2-hydroxypropyl, hydroxybutyl, of ethanyl-1,2-diol, 2-(hydroxymethyl)propanyl-1,3-diol, butanyl-1,2,3,4-tetrol, (2R,4R)-pentanyl-1,2,3,4,5-pentol, (2R,3R,4R,5S)-hexanyl-1,2,3,4,5-pentol, (2R,3R,4R,5R)-hexanyl-1,2,3,4,5,6-hexol, cyclopropyl, cyclobutyl, cyclohexyl, bicyclohexyl, aminomethyl, aminoethyl and benzylaminoethyl.

In another preferred embodiment, R₁ⁿ⁺ is an ammonium cation of formula III, wherein R₅ is H and the ammonium cation of formula III is the ammonium cation derived from an amine of formula V by protonation:

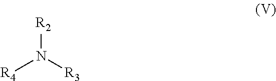

wherein the moieties R₂, R₃ and R₄ are the same in formula III as in formula V, in the present invention, when at least one substituent of the ammonium cation of formula III thereof is H, and at least R₅ is H. More preferably, R₂, R₃ and R₄ are each independently selected from the group consisting of alkyl, hydroxyalkyl, poly(hydroxy)alkyl, aminoalkyl, cycloalkyl, arylakyl, alkylaryl, arylalkylaminoalkyl and alkylaminoaryl.

In a still more preferred embodiment, when R₁ⁿ⁺ is an ammonium cation of formula III and R₅ is H, at least one of R₂, R₃ or R₄ is an alkyl or cycloalkyl. Yet more preferably, said alkyl is a linear alkyl or a branched alkyl moiety. Yet more preferably still, the alkyl or cycloalkyl is selected from the group consisting of methyl, ethyl, propyl, butyl, pentyl, hexyl, cyclopropyl, cyclobutyl, cyclohexyl or bicyclohexyl. Even more preferably, the ammonium cation of formula III is an ammonium cation derived from trimethylamine, propylamine, methylamine, propylamine, 2-propanamine, diethylamine, di(cyclohexyl)amine, cyclobutylamine, cyclopropanemethylamine, 1-bicyclo[1.1.1]pentylamine, meglumine or 2-(dimethylamino)ethanol by protonation. Most preferably, the ammonium cation of formula III is an ammonium cation derived from di(cyclohexyl)amine, meglumine or 2-(dimethylamino)ethanol by protonation.

In another still more preferred embodiment, when R₁ⁿ⁺ is an ammonium cation of formula III and R₅ is H, at least one of R₂, R₃ or R₄ is a linear or branched hydroxyalkyl or a poly(hydroxy)alkyl group. Yet more preferably, when R₅ is H and at least one of R₂, R₃ or R₄ is a linear or branched hydroxyalkyl or a poly(hydroxy)alkyl group, the ammonium cation of formula III is an ammonium cation derived from tromethamine, meglumine or 2-(dimethylamino)ethanol by protonation.

In another still more preferred embodiment, when R₁ⁿ⁺ is an ammonium cation of formula III and R₅ is H, at least one of R₂, R₃, R₄ or R₅ is an aminoalkyl or arylalkylaminoalkyl moiety. Yet more preferably, the aminoalkyl or arylalkylaminoalkyl moiety is selected from an aminoethyl, benzylaminoethyl, aminopropyl, aminoisopropyl or aminobutyl moiety. In an embodiment, when R₁ⁿ⁺ is an ammonium cation of formula III, R₅ is H and at least one of R₂, R₃ or R₄ is an aminoalkyl or arylalkylaminoalkyl moiety, the ammonium cation of formula III is an ammonium cation derived from ethylenediamine, benzathine or diaminopropane by protonation. Most preferably, the ammonium cation of formula III is an ammonium cation derived from ethylenediamine or benzathine by protonation.

In another still more preferred embodiment, when R₁ⁿ⁺ is an ammonium cation of formula III and R₅ is H, at least one of R₂, R₃, R₄ or R₅ is selected from aryl, arylalkyl, alkylaryl, arylalkylaminoalkyl, alkylaminoaryl or aminoaryl, or wherein two of R₂, R₃, R₄ and R₅ are linked to form a heterocyclic group. Yet more preferably, when at least one of R₂, R₃ or R₄ is selected from arylalkyl or arylalkylaminoalkyl, the ammonium cation of formula III is an ammonium cation derived from benzathine by protonation, and when at least one of R₂, R₃ or R₄ is selected from aryl or aminoaryl, or wherein two of R₂, R₃, R₄ and R₅ are linked to form a heterocyclic group, the ammonium cation of formula III is an ammonium cation derived from aniline, pyridine, quinoline or phenylenediamine by protonation.

In another preferred embodiment R₁ⁿ⁺ is an ammonium cation of formula III selected from the group consisting of NH₄_{⁺}, N,N,N-trimethylethanolammonium, and quinolonium.

In another preferred embodiment R₁ⁿ⁺ is an amino acid cation. More preferably, said amino acid cation is derived from an amino acid by protonation, wherein said amino acid is a natural amino acid. Most preferably, the amino acid is L-lysine or L-arginine.

The guanidinium cations of formula IV, as defined in present invention, comprise cations where the charge is either delocalized and cations where the charge is localized in any of the nitrogen atoms, as represented by any of the different canonical representations of formula IV herein below:

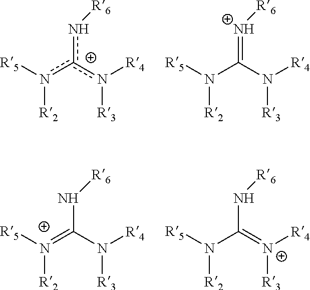

In another preferred embodiment, R₁ⁿ⁺ is a guanidinium cation of formula IV, which is derived from L-arginine by protonation.

In another preferred embodiment, R₁ⁿ⁺ is selected from the group consisting of an alkali metal cation, an alkaline earth metal cation, and a cation derived from L-lysine, L-arginine, trimethylamine, propylamine, methylamine, isopropylamine, butylamine, diethylamine, 2-(dimethylamino)-ethanol, tromethamine, meglumine, cyclobutylamine, cyclopropanemethylamine, dicyclohexylamine, 1-bicyclo[1.1.1]pentylamine, ethylendiamine, diaminopropane, aniline, pyridine, quinoline, phenylenediamine or benzathine by protonation. Where a given cation is disclosed herein as “a cation derived from” a given compound, said cation is that obtained by protonation of the amino or guanidine moiety of said compound. Thus, in another preferred embodiment, R₁ⁿ⁺ is selected from the group consisting of an alkali metal cation, an alkaline earth metal cation, L-lysinate cation, L-arginate cation, trimethyl ammonium cation, propylammonium cation, methylammonium cation, isopropylammonium cation, butylammonium cation, diethylammonium cation, 2-hydroxyethyl-dimethyl ammonium cation, (HOCH₂)₃CNH₃_{⁺} cation, N-methyl-N-sorbitylammonium cation, cyclobutylammonium cation, cyclopropanemethylammonium cation, dicyclohexylammonium cation, 1-bicyclo[1.1.1]pentylammonium cation, 2-amino-ethylammonium cation, aminopropylammonium cation, phenylammonium cation, pyridinium cation, quinolinium cation, amino-phenylammonium cation, or a N—[N′-(phenylmethyl)-aminoethyl]-N-(phenylmethyl)ammonium cation, respectively. In a preferred embodiment of the invention, R₁ⁿ⁺ is selected from Na⁺, K⁺, Ca₂₊ or a cation derived from tromethamine, ethylenediamine, L-arginine, L-lysine, 2-(dimethylamine)ethanol, meglumine or benzathine by protonation.

Cannabigerolic acid (CBGA) is the precursor of the compound of formula I and of all the pharmaceutical salts thereof of formula II of present invention.

The present invention relates to a process to obtain a compound of formula I, as described herein:

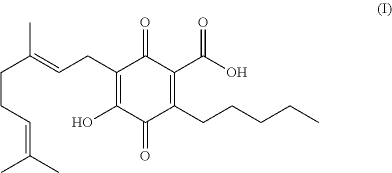 [SMILES: CCCCCC1=C(C(=O)O)C(=O)C(C/C=C(\C)CCC=C(C)C)=C(O)C1=O]

wherein said process comprises the steps of:
a. oxidizing cannabigerolic acid (CBGA) with an oxidizing agent in an aprotic solvent, in the presence of a base having a pKa of at least 11.5, wherein said pKa is measured in water at 25° C., to obtain a compound of formula I:

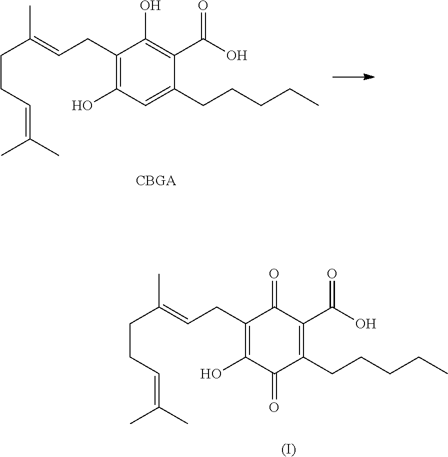

and
b. isolating the compound of formula I formed in step (a).

In a preferred embodiment said aprotic solvent is toluene, acetonitrile, tetrahydrofuran, 1,4-dioxane, dimethylformamide, dimethylsulfoxide, 2-methyltetrahydrofuran or ethyl acetate. In a preferred embodiment the suitable solvent of step (a) is an ether or an ester solvent. More preferably, the ether solvent is tetrahydrofuran or dioxane and the ester solvent is ethyl acetate.

In a preferred embodiment the oxidizing agent is selected from consisting of chlorite, nitrate, periodate, tungstate or air. More preferably, the oxidizing agent is sodium chlorite, sodium periodate, ammonium cerium (IV) nitrate, sodium tungstate dihydrate or air. Most preferably the oxidizing agent is air.

The pKa values, as referred in present application, are measured in water at 25° C., preferably by potentiometric titration, spectrometry, voltammetry, conductometry or electrophoresis.

In a preferred embodiment the base used in step (a) has a pKa of at least 14, more preferably of at least 15, most preferably of about 15 to 38. In a more preferred embodiment, the base used in step (a) is an alkoxide, an alkaline amide base or an alkaline alkylsilylamide base. In an even more preferred embodiment, the base used in step (a) is an alkoxide or an alkaline alkylsilylamide. For the purposes of present invention, the term alkoxide refers to a base comprising an anion RO⁻, wherein R is an alkyl group. Examples of suitable alkoxides include but are not limited to lithium, sodium or potassium alkoxides, in particular lithium, sodium or potassium methoxide, ethoxide, iso-propoxide, propoxide, butoxide, tert-butoxide. Preferably, the alkoxide used in step (a) is lithium, sodium or potassium tert-butoxide, most preferably, potassium tert-butoxide.

For the purposes of present invention, the term alkaline amide base refers to an alkaline azanide base comprising an anion R₂N—, wherein R may be H or an alkyl group. Examples of suitable alkaline amide bases include but are not limited to lithium diethylamide or lithium diisopropylamide.

For the purposes of present invention, the term alkaline alkylsilylamide base, refers to an azanide base comprising the anion R₂N⁻, wherein R is H or an alkylsilyl group. Preferably the alkylsilylamide is sodium bis(trimethylsilyl)amide, potassium bis(trimethylsilyl)amide or lithium bis(trimethylsilyl)amide, most preferably sodium bis(trimethylsilyl)amide or potassium bis(trimethylsilyl)amide.

In a particularly preferred embodiment, the step (a) comprises oxidizing cannabigerolic acid (CBGA) with air, in the presence of an alkoxide and an ether solvent.

Preferably, step (a) is carried out between 15° C. to 25° C. for at least 1 hour. More preferably for 1 to 10 hours, most preferably for 2 to 5 hours.

The present invention also relates to a process to obtain a pharmaceutical salt of formula II, as disclosed herein:

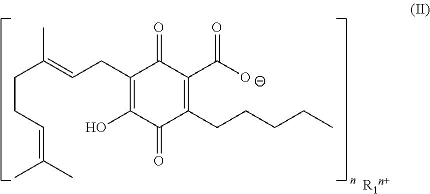

wherein R₁ⁿ⁺ is selected from the group consisting of:
a metal cation;
an amino acid cation;
an ammonium cation of formula III:

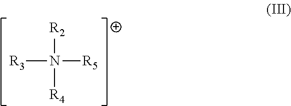

wherein R₂, R₃, R₄ and R₅ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or two of R₂, R₃, R₄ and R₅ are linked to form a heterocyclic group; and
a guanidinium cation of formula (IV):

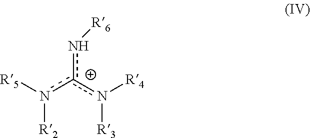

wherein R′₂, R′₃, R′₄, R′₅ and R′₆ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or wherein two of R′₂, R′₃, R′₄, R′₅ and R′₆ are linked to form a heterocyclic group; and

wherein said process comprises:

i. when R₁ⁿ⁺ is a metal cation:
i.a. contacting a solution of the compound of formula I with said metal cation; or
i.b. contacting a solution of the compound of formula I with a first cation to form a salt of the compound of formula I and said first cation; and contacting said salt of the compound of formula I and said first cation with said metal cation;
i.c. contacting a solution of the compound of formula I with the metal from which said metal cation is derived or an inorganic compound of said metal;

ii. when R₁ⁿ⁺ is an amino acid cation:
ii.a contacting a solution of the compound of formula I with said amino acid cation; or
ii.b. contacting a solution of the compound of formula I with a first cation to form a salt of the compound of formula I and said first cation; and contacting said salt of the compound of formula I and said first cation with said amino acid cation; or
ii.c contacting a solution of the compound of formula I with the amino acid from which said amino acid cation is derived by protonation;

iii. when R₁ⁿ⁺ is an ammonium cation of formula III:
iii.a. contacting a solution of the compound of formula I with said ammonium cation of formula III; or
iii.b contacting as solution of the compound of formula I with a first cation to form a salt of the compound of formula I and said first cation; and contacting said salt of the compound of formula I and said first cation with said ammonium cation of formula III; or
iii.c. and when R₅ is H, contacting a solution of the compound of formula I with the amine of formula V from which said ammonium cation of formula III is derived by protonation:

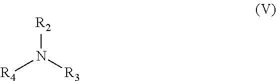

and

iv. when R₁ⁿ⁺ is a guanidinium cation of formula IV:
iv.a. contacting the compound of formula I with said guanidinium cation of formula IV;
iv.b. contacting as solution of the compound of formula I with a first cation to form a salt of the compound of formula I and said first cation; and contacting said salt of the compound of formula I and said first cation with said guanidinium cation of formula IV; or
iv.c. contacting a solution of the compound of formula I with a guanidine derivative of formula IVb from which said guanidium cation of formula IV is derived by protonation

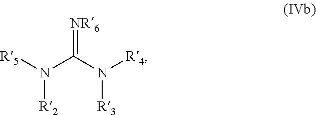

wherein n is a number selected from the group consisting of: 1, 2, 3 and 4.

The first cation, as defined in (i.b), (ii.b), (iii.b) and (iv.b) refers to any cation, which may be an alkali metal cation, an alkaline earth metal cation, a transition metal cation or an organic cation, such as an ammonium cation or a guanidine cation, preferably an alkali metal cation, an alkaline earth metal cation or a transition metal cation. In this sense, (i.b), (ii.b), (iii.b) and (iv.b) provide the pharmaceutical salt of formula II in two sub-steps, in a first sub-step a salt of the compound of formula I and a first cation is formed, and in the second sub-step the first cation is replaced by the cation R₁⁺ⁿ to form the pharmaceutical salt of formula II. Said replacement is performed by ion exchange using a salt of the cation R₁⁺ⁿ and a first anion, wherein said first anion is preferably a halide anion, acetate anion, lactate anion, benzoate anion, triflate anion (CF₃SO₃—), mesylate anion (CH₃SO₃—), thiocyanate anion (SCN—), tBu₂PO₄— anion, PF₆— anion, F₄B— or Ph₄B— anion, More preferably a halide anion selected from a Cl— anion, Br— anion or I— anion or an acetate anion, triflate anion, mesylate anion, PF₆— anion or F₄B— anion.

In (i.c) a metal from which said metal cation is derived, refers to the reduced form of the metal cation. Preferably, said metal is an alkali metal or an alkaline earth metal.

In (i.c) an inorganic compound of a metal cation refers to a compound not including C—C or C—H bonds, which comprises the metal cation by ionic bond. Preferably, said inorganic compound of a metal cation may be selected from the group consisting of a hydroxide, oxide, carbonate, phosphate, sulfate, hydrochloride and hydrobromide.

In a preferred embodiment, said process comprises:
i.a. when R₁ⁿ⁺ is a metal cation, contacting a solution of the compound of formula I with said metal cation;
ii.c. when R₁ⁿ⁺ is an amino acid cation, contacting a solution of the compound of formula I with the amino acid from which said amino acid cation is derived by protonation;
iii. when R₁ⁿ⁺ is an ammonium cation of formula III:
   iii.a. contacting a solution of the compound of formula I with said ammonium cation of formula III; or
   iii.b contacting as solution of the compound of formula I with a first cation to form a salt of the compound of formula I and said first cation; and contacting said salt of the compound of formula I and said first cation with said ammonium cation of formula III; or
   iii.c. and when R₅ is H, contacting a solution of the compound of formula I with the amine of formula V from which said ammonium cation of formula III is derived by protonation:

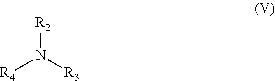

iv.c. when R₁ⁿ⁺ is a guanidinium cation of formula IV, contacting a solution of the compound of formula I with the guanidine derivative of formula IVb from which said guanidium cation of formula IV is derived by protonation:

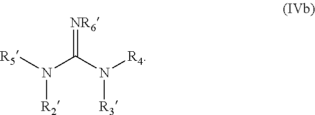

In a particularly preferred embodiment of the process to obtain a pharmaceutical salt of formula II as described herein, comprises contacting a compound of formula I with a compound selected from the group consisting of sodium hydroxide, potassium hydroxide, calcium hydroxide, L-lysine, L-arginine, tromethamine, ethylenediamine, L-arginine, L-lysine, 2-(dimethylamino)-ethanol, dicyclohexylamine, meglumine and benzathine, in equimolar amounts.

In a more particularly preferred embodiment, R₁ⁿ⁺ is a metal cation and said metal cation used in step (i.a) is comprised in a compound selected from sodium hydroxide, potassium hydroxide or calcium hydroxide.

In another more particularly preferred embodiment of the process to obtain a pharmaceutical salt of formula II, R₁ⁿ⁺ is an amino acid cation and the amino acid used in step (ii.c) is a natural amino acid. Still more preferably, said amino acid is L-lysine or L-arginine.

In another more particularly preferred embodiment of the process to obtain a pharmaceutical salt of formula II, R₁ⁿ⁺ is an ammonium cation of formula III, and said ammonium cation of formula III is an ammonium cation derived from an amine of formula V by protonation, as described herein, which is selected from the group consisting of: alkylamine, arylamine, alkyl diamine, arylalkyl dialkylmine, cycloalkylamine, hydroxyalkylamine or poly(hydroxy)alkylamine. More preferably still, said amine of formula V is selected from the group consisting of: tromethamine, meglumine, 2-(dimethylamino)-ethanol, dicyclohexylamine, ethylenediamine and benzathine.

In another more particularly preferred embodiment of the process to obtain a pharmaceutical salt of formula II, R₁ⁿ⁺ is a guanidinium cation, and the guanidine derivative of formula (IVb) used in step (iv.c) is L-arginine.

Preferably, the solution used in each of the embodiments of the process to obtain a pharmaceutical salt of formula II described herein comprises a solvent selected from the group consisting of water, an alcohol, an ether or an ester. Most preferably said solvent is selected from one or more of: ethyl acetate, isopropanol, ethanol, methanol and ethyl ether.

The present invention also relates to a compound of formula I obtainable by the process to obtain a compound of formula I or, to a pharmaceutical salt of formula II obtainable by the process to obtain a pharmaceutical salt of formula II, as described above herein.

Another embodiment of present invention refers to a pharmaceutical composition comprising an effective amount of said compound of formula I and at least one pharmaceutical excipient or carrier.

A further embodiment of present invention refers to a pharmaceutical composition comprising an effective amount of said pharmaceutical salt of formula II and at least one pharmaceutically acceptable excipient or carrier.

As will be inferred below from the examples and figures, the compound of formula I and of all the pharmaceutical salts of formula II thereof, of the present invention, present the capacity to activate PPARγ.

The present invention also relates compounds of formula I or said pharmaceutical salts thereof of formula II, or pharmaceutical compositions comprising said compound of formula I or said pharmaceutical salts thereof of formula II for use as a medicament.

Moreover, the present invention relates to said compound of formula I or said pharmaceutical salt thereof of formula II, or a pharmaceutical composition comprising said compound of formula I or said pharmaceutical salt thereof of formula II, for use in the treatment or prevention of a disease responsive to PPARγ agonists. Diseases responsive to PPARγ agonists are diseases the treatment of which benefits from the administration of said PPARγ agonists. Preferably, the diseases responsive to PPARγ are selected from the group consisting of: atherosclerosis, inflammatory bowel diseases, rheumatoid arthritis, liver fibrosis, nephropathy, psoriasis, skin wound healing, skin regeneration, pancreatitis, gastritis, neurodegenerative disorders, neuroinflammatory disorders, scleroderma, cancer, hypertension, obesity and Type II diabetes. Preferably, said compound of formula I or said pharmaceutical salt thereof of formula II, or a pharmaceutical composition comprising said compound of formula I or said pharmaceutical salt thereof of formula II, is for use as a PPARγ agonist of a PPARγ receptor which does not induce Nfr2 activation. Preferably, said medicament is for use in the treatment of diseases such as atherosclerosis, inflammatory bowel diseases, rheumatoid arthritis, liver fibrosis, nephropathy, psoriasis, skin wound healing, skin regeneration, pancreatitis, gastritis, neurodegenerative disorders, neuroinflammatory disorders, scleroderma, cancer, hypertension, obesity, type II diabetes, and other diseases that can be treated with PPARγ agonists.

Other embodiment of the present invention relates to the use of said compound of formula I or said pharmaceutical salt thereof of formula II in the manufacture of a composition having reduced cytotoxicity for treating PPARγ related diseases such as atherosclerosis, inflammatory bowel diseases, rheumatoid arthritis, liver fibrosis, nephropathy, psoriasis, skin wound healing, skin regeneration, pancreatitis, gastritis, neurodegenerative disorders, neuroinflammatory disorders, scleroderma, cancer, hypertension, obesity, type II diabetes, and other diseases that can be treated with PPARγ agonists.

Analogously, the present invention relates to a method for treating or preventing a disease responsive to PPARγ agonists comprising administering to a patient an effective amount of said compound of formula I or said pharmaceutical salt thereof of formula II, as described herein. Similarly, the present invention relates to a method for treating or preventing a disease responsive to PPARγ agonists comprising administering to a patient an effective amount of said composition comprising said compound of formula I or said pharmaceutical salt thereof of formula II, as described herein, and at least one excipient.

An alternative embodiment of the present invention relates to the use of said compound of formula I or said pharmaceutical salt thereof of formula II, alone or formulated in compositions, particularly pharmaceutical compositions, that comprise at least said compound of formula I or said pharmaceutical salt thereof of formula II, combined with at least one other active compound having additive or synergistic biological activities. Alternatively said compositions can be formulated with at least one inert ingredient as a carrier or excipient such as: cosolvents, surfactants, oils, humectants, emollients, preservatives, stabilizers and antioxidants. Any pharmacologically acceptable buffer may be used, e.g., TRIS or phosphate buffers.

For the purposes of present description, the term “active compound” means a chemical entity or active principle which exerts therapeutic effects when administered to a human or an animal. Typical compositions include said compound of the invention, or or said pharmaceutical salt thereof of formula II, in association with at least one pharmaceutically acceptable excipient, which may be a carrier or a diluent, by a way of example. Such compositions can be in the form of a capsule, sachet, paper or other container. In making the compositions, conventional techniques for the preparation of pharmaceutical compositions may be used. For example, the compound of interest will usually be mixed with a carrier, or diluted by a carrier, or enclosed within a carrier that may be in the form of an ampoule, capsule, sachet, paper, or other container. When the carrier serves as a diluent, it may be solid, semi-solid, or liquid material that acts as a vehicle, excipient, or medium for the active compound. The compound of interest can be adsorbed on a granular solid container for example in a sachet. Some examples of suitable carriers are water, salt solutions, alcohols, polyethylene glycols, polyhydroxyethoxylated castor oil, peanut oil, olive oil, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or colouring substances and the like, which do not deleteriously react with the active compounds.

One preferred embodiment of the present invention refers to the route of administration, that may be any route which effectively transports the compound of interest to the appropriate or desired site of action, such as oral, buccal, nasal, topical, pulmonary, transdermal, parenteral, rectal, subcutaneous, intravenous, intraurethral, intramuscular, intranasal, ophthalmic or ocular.

For nasal administration, the preparation may contain the compound of interest dissolved or suspended in a liquid carrier, in particular an aqueous carrier, for aerosol application. The carrier may contain additives such as solubilizing agents, e.g., propylene glycol, surfactants, absorption enhancers such as lecithin (phosphatidylcholine), or cyclodextrin, or preservatives such as parabens.

To prepare topical formulations, the compound of interest is placed in a dermatological vehicle as is known in the art. The amount of the compound of interest to be administered and the compound's concentration in the topical formulations depend upon the vehicle, delivery system or device selected, the clinical condition of the patient, the side effects and the stability of the compound in the formulation. Thus, the physician employs the appropriate preparation containing the appropriate concentration of the compound of interest and selects the amount of formulation administered, depending upon clinical experience with the patient in question or with similar patients.

For ophthalmic applications, the compound of interest is formulated into solutions, suspensions, and ointments appropriate for use in the eye. The concentrations are usually as discussed above for local preparations.

For oral administration, either solid or fluid unit dosage forms can be prepared. For preparing solid compositions such as tablets, the compound of interest is mixed into formulations with conventional ingredients such as talc, magnesium stearate, dicalcium phosphate, magnesium aluminum silicate, calcium sulfate, starch, lactose, acacia, methylcellulose, and functionally similar materials as pharmaceutical diluents or carriers.

Capsules are prepared by mixing the compound of interest with an inert pharmaceutical diluent and filling the mixture into a hard gelatin or hydroxypropylmethyl cellulose (HPMC) capsule of appropriate size. Soft gelatin capsules are prepared by machine encapsulation of slurry of the compound of interest with an acceptable vegetable oil, light liquid petrolatum or other inert oil. Fluid unit dosage forms for oral administration such as syrups, elixirs and suspensions can be prepared. The water-soluble forms can be dissolved in an aqueous vehicle together with sugar, aromatic flavoring agents and preservatives to form syrup. An elixir is prepared by using a hydroalcoholic (e.g., water/ethanol) vehicle with suitable sweeteners such as sugar and saccharin, together with an aromatic flavoring agent. Suspensions can be prepared with an aqueous vehicle with the aid of a suspending agent such as acacia, tragacanth, methylcellulose and the like.

Appropriate formulations for parenteral use are apparent to the practitioner of ordinary skill, such as the use of suitable injectable solutions or suspensions. The formulation, which is sterile, is suitable for various topical or parenteral routes including intradermal, intramuscular, intravascular, and subcutaneous.

In addition to the compound of interest, the compositions may include, depending on the formulation and mode of delivery desired, pharmaceutically-acceptable, non-toxic carriers or diluents, which include vehicles commonly used to form pharmaceutical compositions for animal or human administration. The diluent is selected so as not to unduly affect the biological activity of the combination.

Examples of such diluents that are especially useful for injectable formulations are water, the various saline, organic or inorganic salt solutions, Ringer's solution, dextrose solution, and Hank's solution. In addition, the pharmaceutical composition or formulation may include additives such as other carriers; adjuvants; or non-toxic, non-therapeutic, non-immunogenic stabilizers and the like.

Furthermore, excipients can be included in the formulation. Examples include cosolvents, surfactants, oils, humectants, emollients, preservatives, stabilizers and antioxidants. Any pharmacologically acceptable buffer may be used, e.g., tris or phosphate buffers. Effective amounts of diluents, additives, and excipients are those amounts that are effective to obtain a pharmaceutically acceptable formulation in terms of solubility or biological activity.

The compound of interest may be incorporated into a microsphere. The compound of interest can be loaded into albumin microspheres, from which it is possible to recover such microspheres in a dry powder for nasal administration. Other materials suitable for the preparation of microspheres include agar, alginate, chitosan, starch, hydroxyethyl starch, albumin, agarose, dextran, hyaluronic acid, gelatin, collagen, and casein. The microspheres can be produced by various processes known to the person skilled in the art such as a spray drying process or an emulsification process.

For example, albumin microspheres can be prepared by adding rabbit serum albumin in phosphate buffer to olive oil with stirring to produce water in oil emulsion. Glutaraldehyde solution is then added to the emulsion and the emulsion stirred to cross-link the albumin. The microspheres can then be isolated by centrifugation, the oil removed, and the spheres washed, e.g., with petroleum ether followed by ethanol. Finally, the microspheres can be sieved and collected and dried by filtration.

Starch microspheres can be prepared by adding a warm aqueous starch solution, e. g., of potato starch, to a heated solution of polyethylene glycol in water with stirring to form an emulsion. When the two-phase system has formed (with the starch solution as the inner phase) the mixture is then cooled to room temperature under continued stirring whereupon the inner phase is converted into gel particles. These particles are then filtered off at room temperature and slurred in a solvent such as ethanol, after which the particles are again filtered off and laid to dry in air. The microspheres can be hardened by well-known cross-linking procedures such as heat treatment or by using chemical cross-linking agents. Suitable agents include dialdehydes, including glyoxal, malondialdehyde, succinicaldehyde, adipaldehyde, glutaraldehyde and phthalaldehyde, diketones such as butadione, epichlorohydrin, polyphosphate, and borate. Dialdehydes are used to cross-link proteins such as albumin by interaction with amino groups, and diketones form Schiff bases with amino groups. Epichlorohydrin activates compounds with nucleophiles such as amino or hydroxyl to an epoxide derivative.

Another preferred embodiment of the invention is the dosage scheme. The term “unit dosage form” refers to physically discrete units suitable as unitary dosages for subjects, e.g., mammalian subjects such as humans, dogs, cats, and rodents, each unit containing a predetermined quantity of active material calculated to produce the desired pharmaceutical effect in association with the required pharmaceutical diluent, carrier or vehicle. The specifications for the unit dosage forms of this invention are dictated by and dependent on (a) the unique characteristics of the active material and the particular effect to be achieved and (b) the limitations inherent in the art of compounding such an active material for use in humans and animals. Examples of unit dosage forms are tablets, capsules, pills, powder packets, wafers, suppositories, granules, cachets, teaspoonfuls, tablespoonfuls, dropperfuls, ampules, vials, aerosols with metered discharges, segregated multiples of any of the foregoing, and other forms as herein described. The compositions can be included in kits, which can contain one or more unit dosage forms of the composition and instructions for use to treat one or more of the disorders described herein. Slow or extended-release delivery systems, including any of a number of biopolymers (biological-based systems), systems employing liposomes, colloids, resins, and other polymeric delivery systems or compartmentalized reservoirs, can be utilized with the compositions described herein to provide a continuous or long-term source of therapeutic compound. Such slow release systems are applicable to formulations for delivery via topical, intraocular, oral, and parenteral routes. They may also be manufactured in the form of sterile solid compositions, such as lyophilized compositions, which can be dissolved or suspended in sterile injectable medium immediately before use. They may contain, for example, suspending or dispersing agents known in the art. An effective amount of the compound of interest is employed in treatment. The dosage of compounds used in accordance with the invention varies depending on the compound and the condition being treated for example the age, weight, and clinical condition of the recipient patient. Other factors include: the route of administration, the patient, the patient's medical history, the severity of the disease process, and the potency of the particular compound. The dose should be sufficient to ameliorate symptoms or signs of the disease treated without producing unacceptable toxicity to the patient. In general, an effective amount of the compound is that which provides either subjective relief of symptoms or an objectively identifiable improvement as noted by the clinician or other qualified observer.

A last embodiment of the present invention relates to methods for treating diseases such as atherosclerosis, inflammatory bowel diseases, rheumatoid arthritis, liver fibrosis, nephropathy, psoriasis, skin wound healing, skin regeneration, pancreatitis, gastritis, neurodegenerative disorders, neuroinflammatory disorders, scleroderma, cancer, hypertension, obesity and Type II diabetes, which can be treated with PPARv agonists, or are responsive to PPARv agonists; that comprises the administration to a patient of an effective amount of a compound of formula I or of a pharmaceutical salt thereof of formula II, or a composition comprising at least one of a compound of formula I or of a pharmaceutical salt thereof of formula II according to present invention.

### EXAMPLES

The examples of present invention described below aim to illustrate some of the embodiments disclosed without limiting its scope of protection.

### Example 1: Synthesis of the Compound of Formula I and Pharmaceutical Salts Thereof of Formula II

Compound of formula I showed to be unstable in most reaction conditions where it undergoes decarboxylation.

Cannabigerol acid (CBGA, (Z)-3-(3,7-dimethylocta-2,6-dienyl)-2,4-dihydroxy-6-pentylbenzoic acid) (0.995 g; 2.76 mmol, Sigma Aldrich) was dissolved in THF (10 mL) at 20° C. and KOtBu (0,867 g; 7.73 mmol) was added (Scheme 1). The mixture was stirred open to the air for 3 h, and the reaction mixture was dissolved in AcOEt (50 mL) and H₂O (50 mL), layers were separated, and the aqueous layer was washed with AcOEt (50 mL). Organic layers were discarded, and the aqueous layer was acidified to pH=5.5 to 6.0 and extracted with AcOEt (2×50 mL). The acidic organic layers were dried (Na₂SO₄) and concentrated under vacuum to obtain compound 1 as a red oil (691 mg, 67%, the sample contained EtOAc).

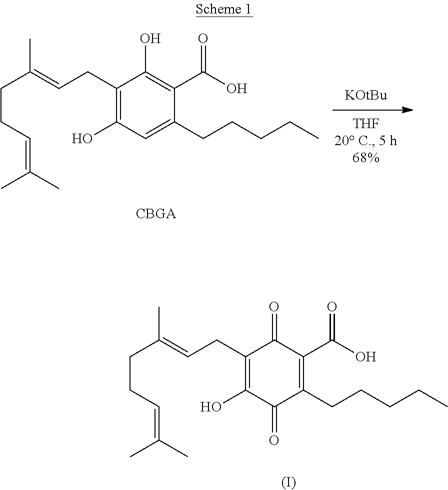

NMR-¹H (CDCl₃, 300 MHz) δ ppm: 5.08 (m, 2H), 3.18 (d, J=7.0 Hz, 2H), 2.80 (t, J=7.6 Hz, 2H), 2.00 (m, 4H), 1.73 (s, 3H), 1.65 (s, 3H), 1.57 (s, 3H), 1.50 (m, 2H), 1.36 (m, 4H), 0.89 (t, J=7.0 Hz, 3H).

MS-ESI-m/z: 373 (M−1, 100); ESI+m/z: 375 (M+1, 70), 392 (M+NH₄, 100)

These conditions yielded compound of formula I with high yields and purity.

Another alternative to synthesize the compound of formula I was explored, conducting the oxidation of CBGA methyl ester disclosed in WO2015128200 (Scheme 1), compound disclosed therein which is the compound closest in structure to compound of formula I. That synthetic route did not provide the acid of formula I. In fact, the oxidation of CBGA methyl ester led to the CBGA quinone ester, but the attempts to hydrolyze it to the desired compound of formula I were not successful. The oxidation reaction of CBGA needs harsh conditions which may produce decarboxylation, obtaining VCE-003 (CBG-Q), instead of the compound of formula I:

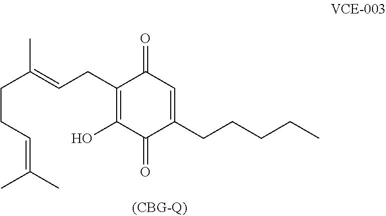 [SMILES: CCCCCC1=CC(=O)C(C/C=C(\C)CCC=C(C)C)=C(O)C1=O]

Said VCE-003, due to the lack of a substitution in the position 2, results in electrophilic (Nrf2 activation) and cytotoxic activities. In this sense, the method described in present invention provides a successful manner to obtain the compound of formula I, in a single synthetic step.

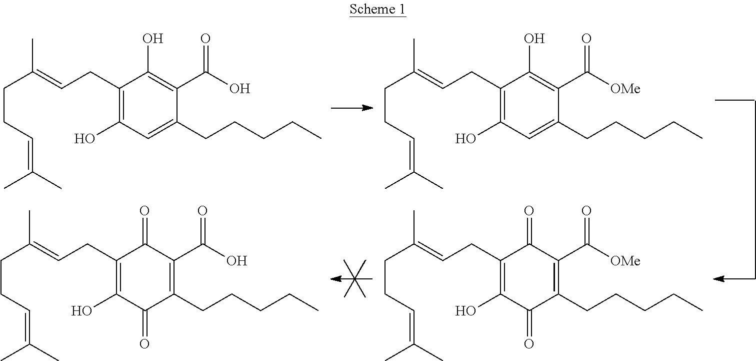

Tromethamine salt (IIa)

Compound of formula I (0.105 g, 0.28 mmol) was dissolved in iPrOH (1 mL). Tromethamine (34 mg; 0.28 mmol, Sigma Aldrich) was added and no precipitate was observed. The mixture was distilled to residue and EtOAc added to obtain a gum oil which was decanted to obtain the tromethamine salt (51 mg, 37%, purity 95.61%).

NMR-¹H (D₂O, 300 MHz) δ ppm: 5.14 (m, 2H), 3.64 (s, 22H), 2.96 (m, 2H), 2.22 (m, 2H), 2.07 (m, 2H), 1.98 (m, 2H), 1.69 (s, 3H), 1.63 (s, 3H), 1.57 (s, 3H), 1.38 (m, 2H), 1.25 (m, 4H), 0.83 (t, J=7.0 Hz, 3H).

L-Lysine Salt (IIi)

Compound of formula I (0.065 g, 0.17 mmol) was dissolved in AcOEt (1 mL). A solution of L-lysine (25 mg, 0.174 mmol, Sigma Aldrich) in water (0.2 mL) was added dropwise. The formed purple oil was decanted, ethanol was added and concentrated under vacuum to remove traces of water and the oil residue was treated with Et₂O (2 mL) to obtain the lysine salt as a dark solid (48 mg, 53%, purity 93.14%).

NMR-¹H (D₂O, 300 MHz) δ ppm: 5.15 (m, 2H), 3.71 (t, J=6.4 Hz, 3H), 3.0 (m, 8H), 2.23 (m, 2H), 2.07 (m, 2H), 1.98 (m, 2H), 1.89 (m, 5H), 1.70 (s, 3H), 1.69 (m, 5H), 1.64 (s, 3H), 1.58 (s, 3H), 1.44 (m, 4H), 1.27 (m, 3H), 0.85 (m, 3H).

L-Arginine Salt (IIg)

Compound of formula I (0.097 g, 0.259 mmol) was dissolved in AcOEt (1 mL). A solution of L-arginine (45 mg; 0.259 mmol, Sigma Aldrich) in water (0.2 mL) was added dropwise and a dark oil was formed. Solvent was removed under vacuum and ethanol (2×3 mL) added and concentrated under vacuum to remove traces of water. The oily residue was treated with Et₂O (2 mL) to obtain the arginine salt as a dark solid (87 mg, 61%, purity 98.23%).

NMR-¹H (D₂O, 300 MHz) δ ppm: 5.13 (m, 2H), 3.74 (t, J=5.9 Hz, 2H), 3.22 (t, J=6.4 Hz, 4H), 2.96 (d, J=5.9 Hz, 2H), 2.24 (m, 2H), 2.06 (m, 2H), 1.97 (m, 2H), 1.88 (m, 6H), 1.69 (s, 3H), 1.68 (m, 3H), 1.63 (s, 3H), 1.56 (s, 3H), 1.40 (m, 2H), 1.27 (m, 4H), 0.84 (m, 3H).

Benzathine Salt (IIc)

Compound of formula I (0.10 g, 0.25 mmol) was dissolved in AcOEt (1 mL). Benzathine (0.067 g; 0.283 mmol, Sigma Aldrich) was added and a slight precipitate was observed. The mixture was cooled down to 0 to 5° C., stirred for 1 hour and filtered to obtain benzathine salt as a dark solid (93 mg, 53%, purity 95.13%).

NMR-¹H (DMSO-d₆, 300 MHz) δ ppm: 7.39 (m, 12H), 5.05 (m, 2H), 3.89 (s, 4H), 2.85 (m, 6H), 2.37 (m, 1H), 1.96 (m, 2H), 1.87 (m, 2H), 1.64 (s, 3H), 1.60 (m, 3H), 1.52 (s, 3H), 1.36 (s, 3H), 1.34 (m, 2H), 1.23 (m, 4H), 0.83 (m, 3H).

Ethylenediamine Salt (IIb)

Compound of formula I (0.099 g, 0.264 mmol) was dissolved in AcOEt (1 mL) and ethylenediamine (0.018 mL; 0.264 mmol, Sigma Aldrich) was added dropwise. The formed dark sticky paste was filtered, and the cake was washed with Et₂O (2 mL) to obtain ethylenediamine salt 40 mg as a dark purple solid (40 mg, 35%; 96.46%)

NMR-¹H (DMSO-d₆, 300 MHz) δ ppm: 4.99 (m, 2H), 3.09 (m, 5H), 2.81 (m, 5H), 2.08 (m, 2H), 1.92 (m, 2H), 1.83 (m, 2H), 1.54 (s, 3H), 1.48 (s, 3H), 1.25 (s, 3H), 1.25 (m, 2H), 1.12 (m, 4H), 0.68 (m, 3H).

Meglumine Salt (IIh)

Compound of formula I (0.106 g, 0.283 mmol) was dissolved in MeOH (1 mL). Meglumine (55 mg; 0.283 mmol, Sigma Aldrich) was added. No precipitate was observed. The mixture was distilled until residue and AcOEt (1 mL) was added to obtain an oil, which was decanted and washed with Et₂O (1 mL). The oil became to a dark solid under drying at high vacuum (43 mg, 27%; purity 99.29%).

Sodium Salt (IIe)

Compound of formula I (0.086 g, 0.23 mmol) was dissolved in AcOEt (1 mL). A solution of NaOH 1 N (0.045 mL, 0.23 mmol, Sigma Aldrich) was added and the mixture was distilled to residue and slurried in AcOEt (1 mL), stirred at room temperature and filtered to obtain the sodium salt as a brownish solid (18 mg, 20%, purity 99.34%).

NMR-¹H (CDCl₃, 300 MHz) δ ppm: 5.04 (m, 2H), 3.04 (d, J=7.0 Hz, 2H), 2.40 (t, J=8.2 Hz, 2H), 2.00 (m, 2H), 1.79 (m, 2H), 1.67 (s, 3H), 1.63 (s, 3H), 1.55 (s, 3H), 1.47 (m, 2H), 1.28 (m, 4H), 0.84 (t, J=7.0 Hz, 3H).

Potassium Salt (IIj)

Compound of formula I (0.106 g, 0.283 mmol) was dissolved in Et₂O (1 mL). A solution of KOH (0.016 g, 0.24 mmol, Sigma Aldrich) in water (0.05 mL) was added and an oil was formed. The mixture was concentrated to remove the water and the residue was slurried in Et₂O (2 mL) to obtain the potassium salt as a sticky solid (29 mg, 25%, purity 90.09%). Mother liquors were treated with KOH (7 mg) in methanol/water 5:0.2 (0.5 mL), concentrated and the residue slurried in Et₂O to obtain a second crop of potassium salt (51 mg, 44%, 91.09% purity).

Calcium Salt (IId)

To a solution of compound of formula I (0.099 g, 0.264 mmol) in EtOAc (1 mL) was added dropwise a solution of Ca(OH)₂ (0.020 g, 0.264 mmol, Sigma Aldrich) in water (0.05 mL). The mixture was concentrated to residue and then EtOAc (2×1 mL) was added and concentrated under vacuum to remove traces of water. The residue was slurried in EtOAc (1 mL) for 0.5 hour and filtered to obtain the calcium salt as a dark solid (105 mg, 100%, 98.66%).

NMR-¹H (DMSO-d₆, 300 MHz) δ ppm: 5.10 (m, 2H), 2.08 (d, J=7.0 Hz, 2H), 2.18 (m, 2H), 1.97 (m, 2H), 1.87 (m, 2H), 1.73 (s, 3H), 1.63 (s, 3H), 1.55 (s, 3H), 1.24 (m, 6H), 0.85 (t, J=7.0 Hz, 3H).

Dicyclohexylamine Salt (IIf)

To a solution of compound of formula I (0.105 g, 0.28 mmol) in iPrOH (1 mL) at 0-5° C. was added dicyclohexylamine (0.051 mg; 0.28 mmol, Sigma Aldrich). The mixture was stirred at low temperature for 2 hours and filtered to obtain dicyclohexylamine salt as a dark solid (76 mg, 49% yield; purity 98.74%)

NMR-¹H (CDCl₃, 300 MHz) δ ppm: 5.13 (m, 1H), 5.05 (m, 1H), 3.85 (bs, 4H), 3.09 (d, J=7.0 Hz, 2H), 2.81 (m, 4H), 2.43 (m, 2H), 1.96 (m, 12H), 1.75 (m, 10H), 1.71 (s, 3H), 1.65 (s, 3H), 1.57 (s, 3H), 1.26 (m, 23H), 0.87 (t, J=7.0 Hz, 3H).

### 2-(Dimethylamino)ethanol salt (IIk)

To a solution of compound of formula I (0.097 g, 0.26 mmol) in EtOAc (1 mL) was added 2-(dimethylamino) ethanol (0.023 mg; 0.26 mmol, Sigma Aldrich). The formed oil was decanted and then treated with Et₂O, but a solid was not formed. The mixture was concentrated, and the residue was dried under high vacuum to obtain the title salt as dark oil (59 mg, 49%, purity 95.76%)

NMR-¹H (CDCl₃, 300 MHz) δ ppm: 5.08 (m, 2H), 4.87 (sa, 7H), 3.87 (m, 3H), 3.08 (d, J=7.0 Hz, 2H), 2.98 (m, 3H), 2.73 (s, 9H), 2.42 (m, 2H), 2.01 (m, 2H), 1.93 (m, 2H), 1.70 (s, 3H), 1.65 (s, 3H), 1.57 (s, 3H), 1.50 (m, 2H), 1.31 (m, 4H), 0.87 (m, 3H).

### Example 2: PPARγ Binding Assays

PPARγ binding activity was determined by using PolarScreen™ PPAR Competitor Assay kit (Life Technologies), according to the manufacturer's specifications. The PolarScreen™ PPAR Competitor Assay is a binding assay for determining IC₅₀ values of compounds that bind to PPARγ.

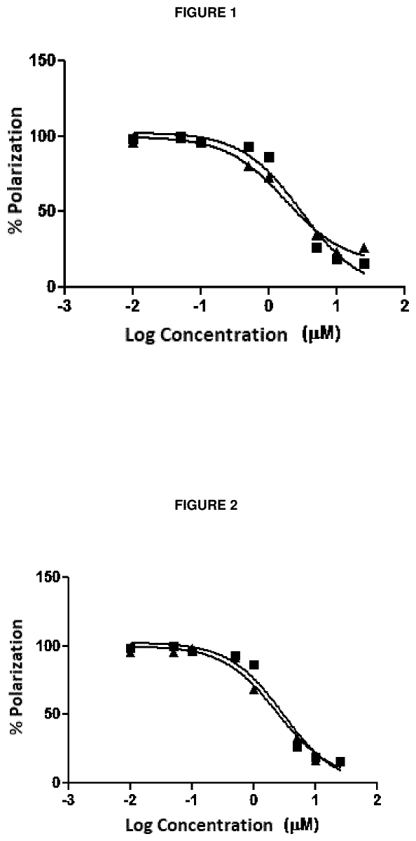

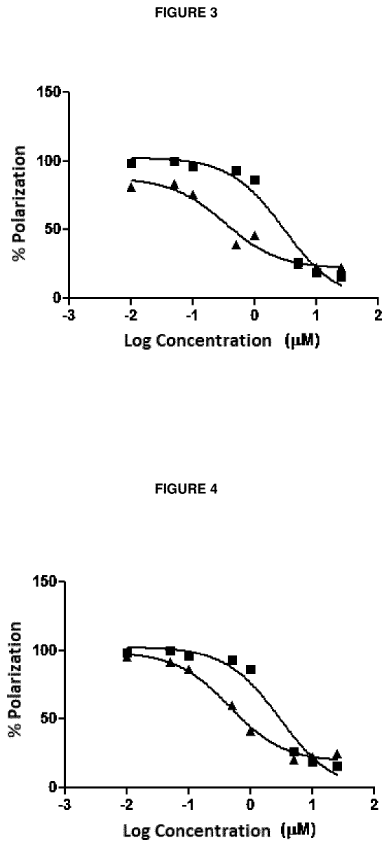

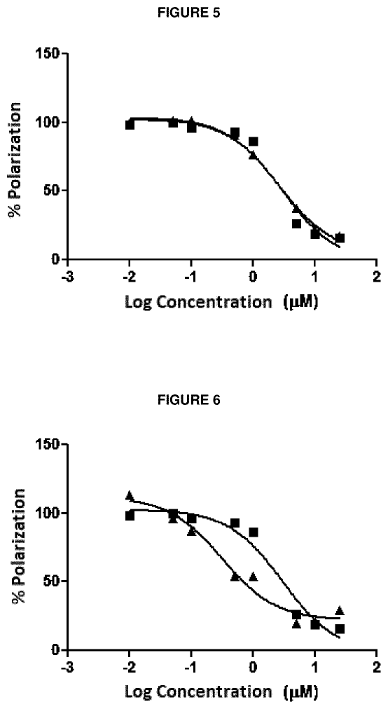

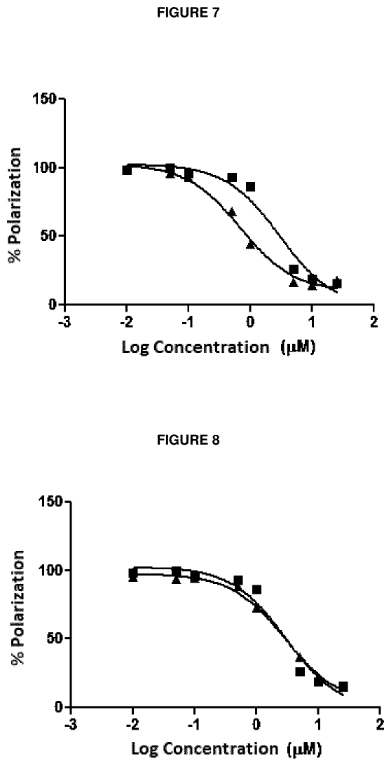

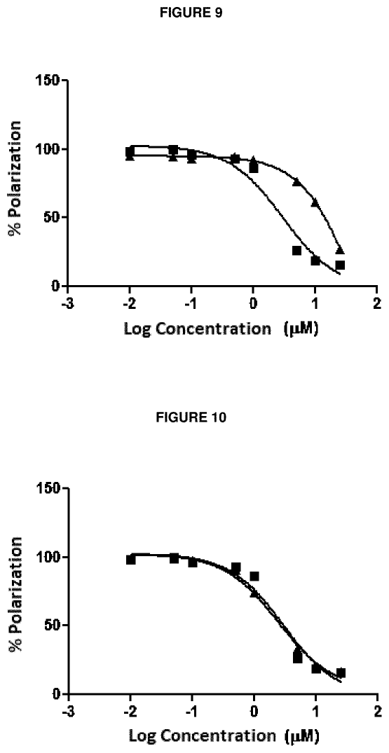

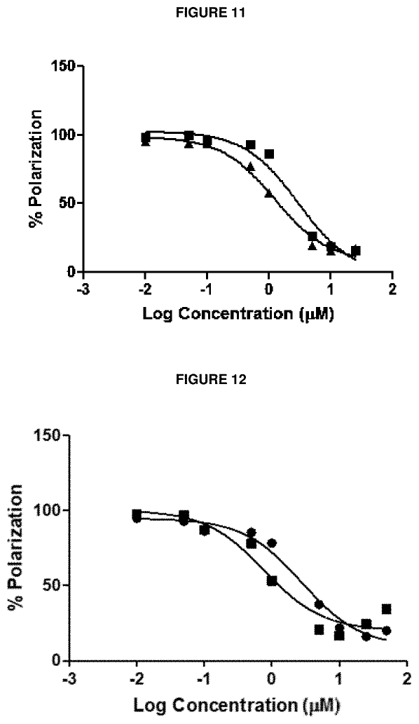

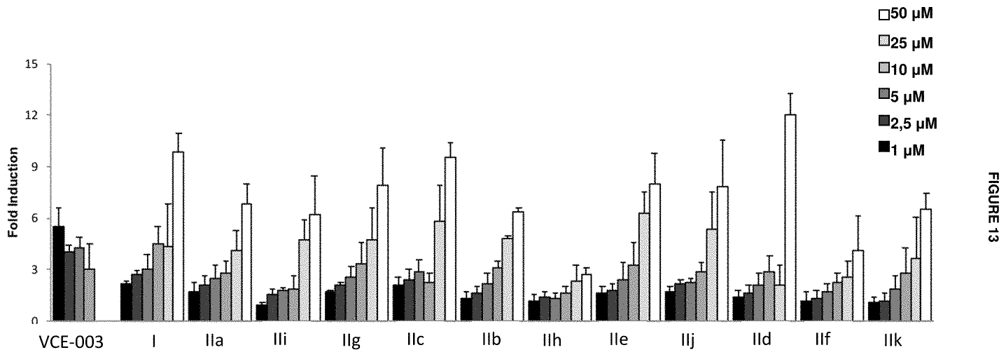

Relative affinity for PPARγ, as percentage of polarization were plotted against the concentration of the compound of formula I and salts of formula II obtained in Example 1 as shown in FIGS. 1 to 12.

Concentration of the compound of formula I and salts of formula II resulting in a half maximal shifts in polarization value determines the IC₅₀. Table 1 includes the IC₅₀ values of compound of formula I (I) and salts of formula II of tromethamine (IIa), ethylenediamine (IIb), benzathine (IIc), calcium (IId), sodium (IIe), dicyclohexylamine (IIf), L-arginine (IIg), meglumine (IIh), L-lysine (IIi), potassium (IIj) and 2-dimethylaminoethanol (IIk) compared to the reference PPARγ agonist VCE-003.

TABLE 1

| ∝M | VCE-003 | | I | IIa | IIb | IIc | IId |
|---|---|---|---|---|---|---|---|
| 0 | 100 | | 100 | 100 | 100 | 100 | 100 |
| 0.01 | 98.1 | | 97.8 | 95.9 | 95.0 | 81.3 | 95.1 |
| 0.05 | 99.5 | | 97.3 | 98.8 | 95.6 | 83.4 | 91.7 |
| 0.1 | 95.9 | | 87.3 | 96.1 | 98.6 | 75.7 | 86.0 |
| 0.5 | 93.3 | | 78.2 | 80.5 | 91.7 | 38.9 | 60.2 |
| 1 | 86.2 | | 53.3 | 73.0 | 68.4 | 45.6 | 41.4 |
| 5 | 26.7 | | 20.7 | 34.9 | 33.2 | 24.9 | 20.7 |
| 10 | 19.1 | | 16.5 | 23.2 | 16.5 | 22.9 | 22.5 |
| 25 | 16.0 | | 24.4 | 26.1 | 16.1 | 22.9 | 24.9 |
| 50 | 24.5 | | 34.4 | 30.9 | 16.1 | 27.0 | 45.4 |
| LOG IC50 | 0.4771 | | −0.3134 | 0.1643 | 0.3188 | −0.5036 | −0.4685 |
| IC50 | 3.000 | | 0.77 | 1.460 | 2.084 | 0.3136 | 0.3400 |
| ∝M | IIe | IIf | IIg | IIh | IIi | IIj | IIk |
| 0 | 100 | 100 | 100 | 100 | 100 | 100 | 100 |
| 0.01 | 99.6 | 113.2 | 99.0 | 95.4 | 95.2 | 99.3 | 95.7 |
| 0.05 | 101.6 | 96.3 | 95.9 | 94.2 | 94.3 | 99.1 | 94.0 |
| 0.1 | 101.6 | 87.4 | 92.8 | 94.9 | 93.3 | 97.9 | 93.6 |
| 0.5 | 90.9 | 54.0 | 68.3 | 88.3 | 94.7 | 90.9 | 77.4 |
| 1 | 76.7 | 54.2 | 44.3 | 73.1 | 92.3 | 74.3 | 57.9 |
| 5 | 37.5 | 19.3 | 16.3 | 37.2 | 76.5 | 33.1 | 19.3 |
| 10 | 21.7 | 20.9 | 14.1 | 20.6 | 6.9 | 19.0 | 15.9 |
| 25 | 17.2 | 29.0 | 17.8 | 15.3 | 27.4 | 16.7 | 17.2 |
| 50 | 19.1 | 104.9 | 22.1 | 13.7 | 15.8 | 19.0 | 23.6 |
| LOG IC50 | 0.3483 | −0.9711 | −0.1569 | 0.4703 | 1.371 | 0.3879 | 0.0988 |
| IC50 | 2.230 | 0.1069 | 0.6968 | 2.953 | 23.51 | 2.443 | 1.256 |

### Example 3: PPARγ Transcriptional Activity

To investigate the biological activities of the acid of formula (I) and salts of formula (II) we performed PPARγ transactivation assays in HEK-293T cells.

HEK293T cells were maintained at 37° C. in a humidified atmosphere containing 5% C02 in DMEM supplemented with 10% fetal calf serum (FBS), and 1% (v/v) penicillin/streptomycin. All reagents were from Sigma Co (St Louis, Mo., USA). HEK293T cells (2×10³/well) were seeded in BD Falcon™ White with Clear Bottom 96-well Microtest™ Optilux™ Plate for 24 hours. Afterwards, cells were transiently co-transfected with the expression vector GAL4-PPARγ and the luciferase reporter vector GAL4-luc using Roti©-Fect (Carl Roth, Karlsruhe, Germany) following the manufacturer's instructions. Twenty-four hours post-transfection, cells were pretreated with increasing doses of the compounds for 6 hours. Then, the cells were lysed in 25 mM Tris-phosphate pH 7.8, 8 mM MgCl₂, 1 mM DTT, 1% Triton X-100, and 7% glycerol. Luciferase activity was measured in the cell lysate using a TriStar LB 941 multimode microplate reader (Berthold) and following the instructions of the Luciferase Assay Kit (Promega, Madison, Wis., USA). Protein concentration was measured by the Bradford assay (Bio-Rad, Richmond, Calif., USA). The background obtained with the lysis buffer was subtracted in each experimental value and the specific transactivation expressed as a fold induction over untreated cells. All the experiments were repeated at least three times. The plasmids used were Gal4-hPPARgamma (plasmid name: pCMV-BD-hPPARγ, made in Sinal Laboratory, Dept. of Pharmacology, Dalhousie University) and Gal4-Luc reporter plasmid that includes five Gal4 DNA binding sites fused to the luciferase gene. The results of the above assay are illustrated by FIG. 13 which shows the effect of CBGA-Q (compound I) and salts of formula II (wherein R₁ⁿ⁺ is a cation derived from a compound selected from: tromethamine, L-lysine, L-arginine, benzathine, ethylenediamine, meglumine, sodium, potassium, calcium, dicyclohexylamine and dimethylamine) on PPARγ activity by means of a transactivation assay performed in cells transiently overexpressing PPARγ in combination with a luciferase reporter gene (PPARγ-GAL4/GAL4-Luc) and treated with the compounds for 6 hours. Data are given as means with deviation standard error bars of three replicates.

### Example 4: Efficacy of Cannabigerol Quinone Derivatives in the 3NP Murine Model of Huntington's Disease

The intoxication of mice with 3-nitropropionic acid (3NP), a potent irreversible inhibitor of mitochondrial complex II enzyme, leads to mitochondrial dysfunction and oxidative stress in animal models that results in a myriad of neurological, biochemical and histological effect that were reminiscent of some aspects of Huntington's Disease (HD) pathology. For example, 3NP-treated mice exhibited high scores in hindlimb clasping, dystonia, kyphosis and in the general locomotor activity compared to control animals.

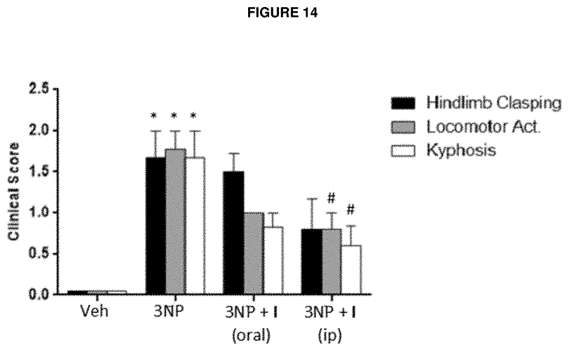

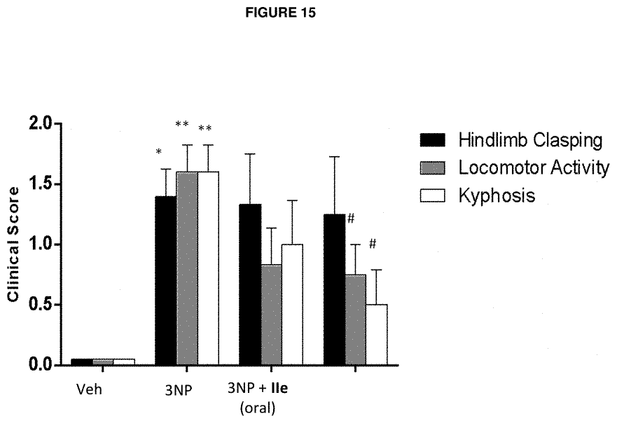

Lesions of the striatum were induced with 3-NP in adult (16-week-old; 30 g) male C57BL/6 mice (Harlan Ibérica, Barcelona, Spain). To this end, mice were subjected to seven intraperitoneal (i.p.) injections of 3NP (one injection each 12 hours) at a dose of 50 mg/kg (prepared in phosphate-buffered saline) for 3 days. These animals and their respective non-lesioned controls were used for pharmacological studies with either cannabigerol quinone acid (I) or with the sodium salt of the cannabigerol quinone acid of formula II. At least 5 to 6 animals were used per experimental group. Treatments consisted of four i.p. injections or oral gavage of the compounds at the indicated doses (one treatment each 24 hours), or vehicle 30 min before the injections of 3NP. All animals were euthanized 12 hours after the last 3NP injection. Once euthanized, animals were dissected, and their brains were rapidly removed. The right hemisphere was used to dissect the striatum, which was quickly frozen in RNAlater (Sigma-Aldrich, Germany) to analyse inflammatory markers by Real Time PCR. The left hemisphere was fixed in fresh 4% paraformaldehyde (in 0.1M phosphate buffered-saline) for 48 hours at 4° C. and embedded in paraffin wax for histological analysis. Mice were subjected to behavioral tests for determining their neurological status. We evaluated the general locomotor activity, the hindlimb clasping and dystonia, and the truncal dystonia. All behavioral tests were conducted prior to drug injections to avoid acute effects of the compounds under investigation. Cannabigerol quinone acid (I) (FIG. 14) and sodium salt of the cannabigerol quinone acid of formula II (FIG. 15) clearly alleviates the clinical symptoms induced by 3-NP intoxication.

### Example 5. Histological Analysis

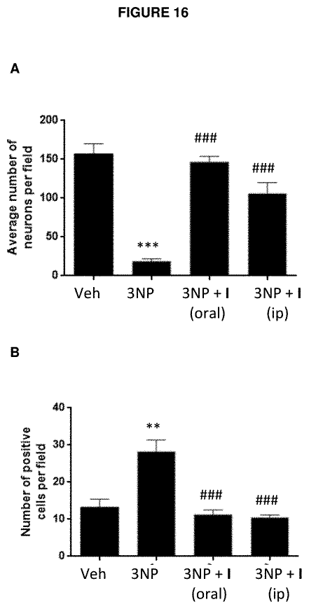

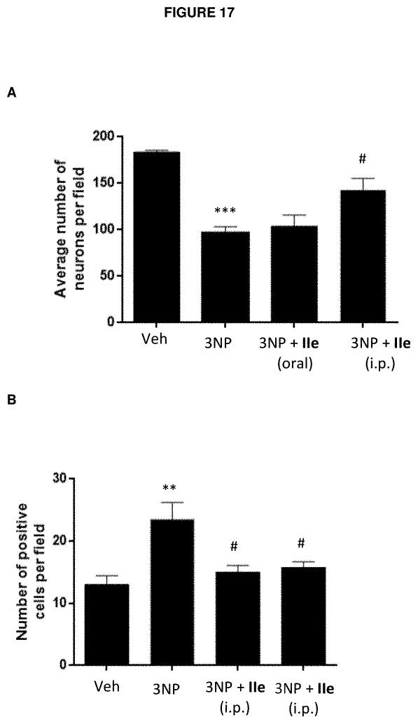

Brains from 3NP model were fixed in 4% paraformaldehyde and 5-μm-thick sections for Nissl staining and immunohistochemical analysis of Iba-1, a marker of microglial cells. For immunohistochemistry sections were incubated overnight at 4° C. with monoclonal anti-mouse Iba-1 antibody (Millipore, Mass., USA) used at 1/50 dilution. After incubation with the corresponding primary antibody, sections were washed in 0.1 M PBS and incubated O/N at 4° C. with goat anti-mouse (Millipore, Mass., USA) secondary antibody. Reaction was revealed with diaminobenzidine. Negative control sections were obtained using the same protocol with omission of the primary antibody. All sections for each immunohistochemical procedure were processed at the same time and under the same conditions. A Leica DM2500 microscope and a Leica DFC 420C camera were used for slide observation and photography, and all image processing was done using ImageJ, the software developed and freely distributed by the US National Institutes of Health (Bethesda. Md., USA). The striatal parenchyma of these 3NP-lesioned animals showed an important degree of neuronal death that was confirmed by Nissl staining. The loss of neurons (Nissl positive cells) was accompanied by a notable increased expression of Iba-1+ cells (reactive microgliosis). Cannabigerol quinone acid (I) (FIG. 16) and sodium salt (IIe) of the cannabigerol quinone acid of formula II (FIG. 17) originated a preservation of striatal neurons against 3NP toxicity as revealed by Nissl staining. Moreover, the treatment with both compounds prevented the induction of reactive microgliosis (Iba-1+ cells).

### Example 6. Real-Time Quantitative PCR Used in the Invention

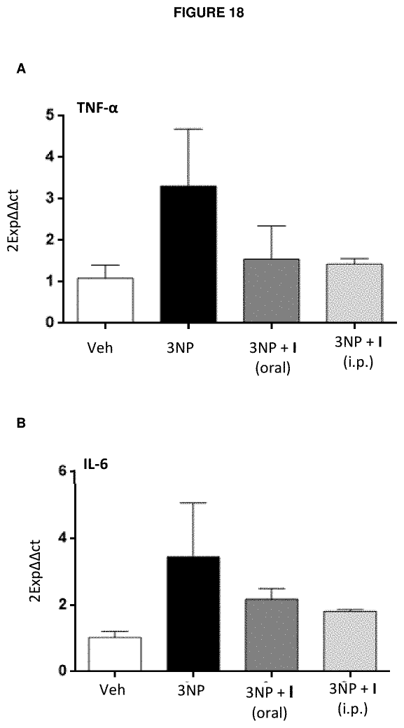

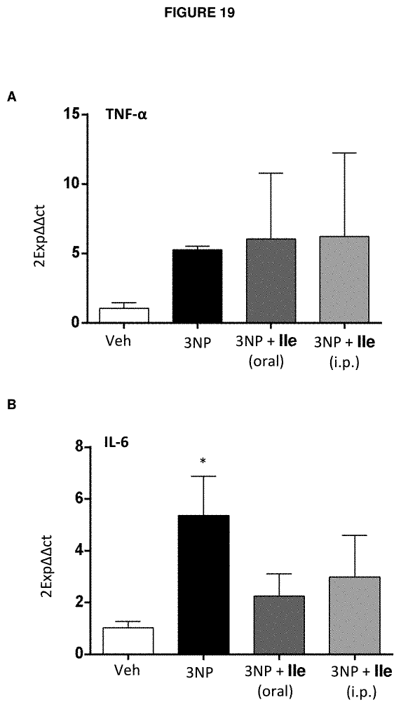

Total RNA was isolated from striata (3NP model) using RNeasy Lipid Tissue Mini Kit (Qiagen, GmbH). The total amount of RNA extracted was quantitated by spectrometry at 260 nm and its purity from the ratio between the absorbance values at 260 and 280 nm. Genomic DNA was removed to eliminate DNA contamination. Single-stranded complementary DNA was synthesized from up to 1 μg of total RNA (pool from at least 3 animals per group) using iScript™ cDNA Synthesis Kit (Bio-Rad, Hercules, Calif., USA). The reaction mixture was kept frozen at −20° C. until enzymatic amplification. The iQ™ SYBR Green Supermix (Bio-Rad) was used to quantify mRNA levels for TNF-α and IL-6. Real-time PCR was performed using a CFX96 Real-Time PCR Detection System (Bio-Rad). The GAPDH housekeeping gene was used to standardize the mRNA expression levels in every sample. Expression levels were calculated using the 2^{-ΔΔCt} method. Sequences of oligonucleotide primers are given in Table 2. The expression of proinflammatory cytokines TNFα and IL-6 was significantly up regulated in 3NP-lesioned mice. Cannabigerol quinone acid (I) (FIG. 18) and sodium salt (IIe) of the cannabigerol quinone acid of formula II (FIG. 19) attenuated the up-regulation of pro-inflammatory markers TNFα and IL-6 in the striatum of mice treated with 3NP. Table 2: List of mouse primer sequences used in quantitative Polymerase Chain Reaction.

TABLE 2

| Genes | Forward | Reverse |
|---|---|---|
| IL-6 | 5′-GAACAACGATGATGCACTTGC-3′ | 5′-TCCAGGTAGCTATGGTACTCC-3′ |
| TNFα | 5′-AGAGGCACTCCCCCAAAAGA-3′ | 5′-CGATCACCCCGAAGTTCCCATT-3′ |
| GAPDH | 5′-TGGCAAAGTGGAGATTGTTGCC-3′ | 5′-AAGATGGTGATGGGCTTCCCG-3′ |

### Example 7. Induction of Parkinson's Disease (6-OHDA Model)

Cannabigerol quinone acid (I) and sodium salt (IIe) of the cannabigerol quinone acid of formula II were also of therapeutic use in a murine model of Parkinson's disease (PD).

C57BL/6 mice pretreated intracerebroventricularly were anesthetized with an intraperitoneal injection of 200 mg/Kg of 2,2,2-tribromoethanol (Sigma-Aldrich) and placed in a stereotaxic frame with a mouse adapter (David Kopf Instruments, Tujunga, Calif., USA). Using a Hamilton syringe (Hamilton, Bonaduz, Switzerland), 4 μL of 6-OHDA-HBr solution (5 μg/pL) in 0.02% ascorbic acid (SigmaAldrich) were injected in the left striatum in two deposits at the following stereotaxic coordinates (mm from bregma): AP, +0.65; L, −2.0; V1, −4 and V2, −3.5, targeting the dorsolateral striatum. After the injection, the skin was sutured, and the animals were removed from the stereotaxic instrument and placed on a heating pad for 30 min. The mice were subjected to chronic oral or intraperitoneal treatment with Cannabigerol quinone acid (I) and sodium salt (IIe) of the cannabigerol quinone acid of formula II, or vehicle starting 16 h after the 6-OHDA injection and for 14 days. Pole and Cylinder rearing tests were used to evaluate motor activity.

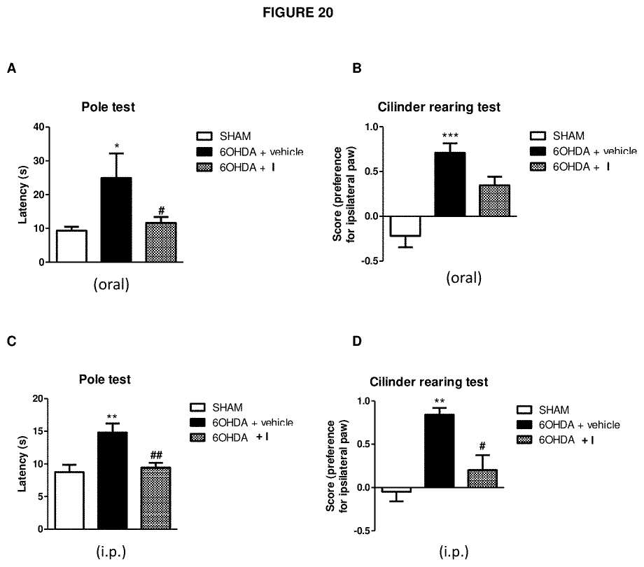

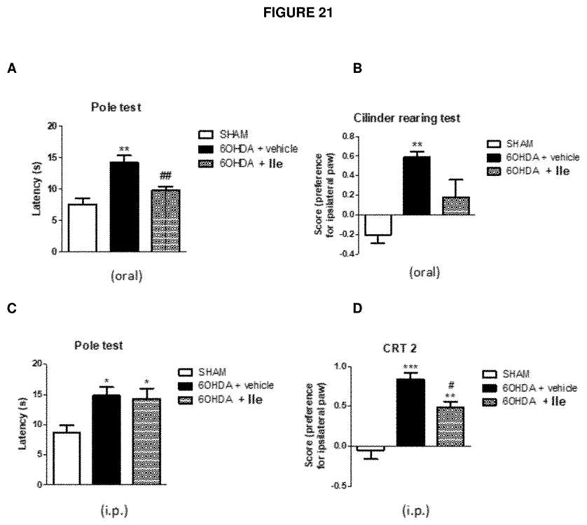

The pole test was used to detect bradykinesia and motor and cylinder rearing test to evaluate sensory-motor deficits in PD mice. For Pole test mice were placed head-upward on the top of a vertical rough-surfaced pole (diameter 8 mm; height 55 cm) and the time until animals descended to the floor was recorded with a maximum duration of 90 s. The trial was made after one training. When the mouse was not able to turn downward and instead dropped from the pole, the time was taken as 90 s (default value). For Cylinder Rearing Test (CRT) the initial forepaw (left, right, or both) preference after placing the mouse in a methacrylate transparent cylinder of 15.5 cm in diameter and 12.7 cm in height was measured. Each score was made out of a 3 min trial with a minimum of 4 wall contacts. Cannabigerol quinone acid (I) (FIG. 20) and sodium salt (IIe) of the cannabigerol quinone acid of formula II (FIG. 21) attenuated bradykinesia and motor deficit in 6-OHDA challenged mice.

1. A compound of formula I: 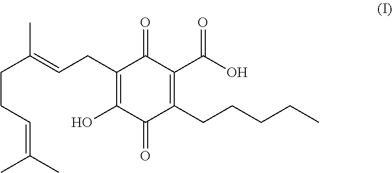 [SMILES: CCCCCC1=C(C(=O)O)C(=O)C(C/C=C(\C)CCC=C(C)C)=C(O)C1=O]
or a pharmaceutical salt of formula II of said compound of formula I: 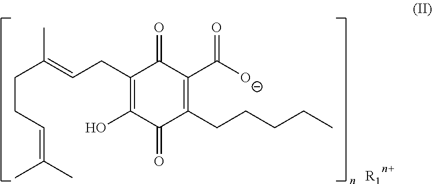
wherein R₁ⁿ⁺ is selected from the group consisting of:
a metal cation;
an amino acid cation;
an ammonium cation of formula III: 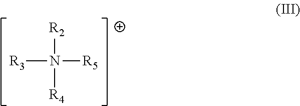
wherein R₂, R₃, R₄ and R₅ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or two of R₂, R₃, R₄ and R₅ are linked to form a heterocyclic group; and
a guanidinium cation of formula (IV): 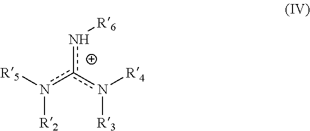
wherein R′₂, R′₃, R′₄, R′₅ and R′₆ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or wherein two of R′₂, R′₃, R′₄, R′₅ and R′₆ are linked to form a heterocyclic group,
wherein n is a number selected from the group consisting of: 1, 2, 3 and 4.

2. A compound according to claim 1, wherein said compound is a compound of formula (I):

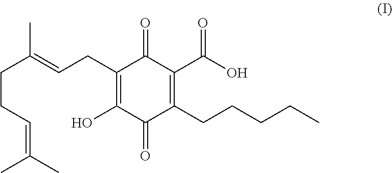 [SMILES: CCCCCC1=C(C(=O)O)C(=O)C(C/C=C(\C)CCC=C(C)C)=C(O)C1=O]

3. A compound according to claim 1, wherein said compound is a pharmaceutical salt of formula (II): 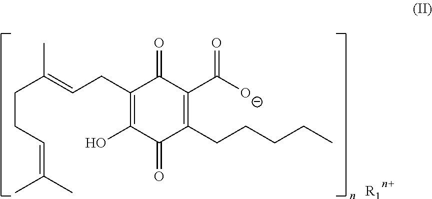
wherein n is a number selected from 1 or 2.

4. A compound according to claim 3, wherein R₁ⁿ⁺ is an alkali metal cation or an alkaline earth metal cation.

5. A compound according to claim 3, wherein R₁ⁿ⁺ is an ammonium cation of formula III, wherein at least one of R₂, R₃, R₄ or R₅ is selected from the group consisting of: alkyl, hydroxyalkyl, poly(hydroxy)alkyl, aminoalkyl, cycloalkyl, arylakyl, alkylaryl, arylalkylaminoalkyl and alkylaminoaryl.

6. A compound according to claim 3, wherein R₁ⁿ⁺ is an amino acid cation.

7. A compound according to claim 3, wherein R₁ⁿ⁺ is selected from the group consisting of: Na⁺, K⁺, Ca²⁺, or a cation of tromethamine, ethylenediamine, L-arginine, L-lysine, 2-(dimethylamino) ethanol, dicyclohexylamine, meglumine and benzathine.

8. A process to obtain a compound of formula I: 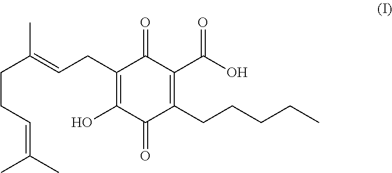 [SMILES: CCCCCC1=C(C(=O)O)C(=O)C(C/C=C(\C)CCC=C(C)C)=C(O)C1=O]
wherein said process comprises the steps of:
a. oxidizing cannabigerolic acid (CBGA) with an oxidizing agent in an aprotic solvent, in the presence of a base having a pKa of at least 11.5, wherein said pKa is measured in water at 25° C., to obtain a compound of formula I: 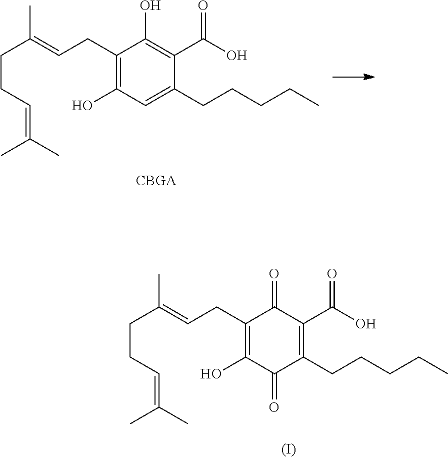
and
b. isolating the compound of formula I.

9. A process according to claim 8, wherein the aprotic solvent of step (a) is an ether or an ester.

10. A process according to claim 8, wherein the oxidizing agent is selected from the group consisting of: chlorite, nitrate, periodate, tungstate and air.

11. A process according to claim 8, wherein the base is an alkali metal alkoxide, an alkaline earth metal alkoxide or an alkali metal alkylsilylamide.

12. A process to obtain a pharmaceutical salt of formula II: 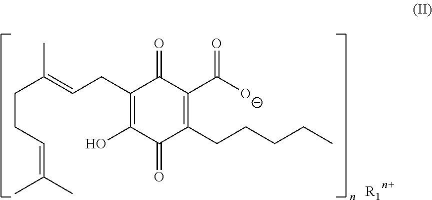
wherein R₁ⁿ⁺ is:
a metal cation;
an amino acid cation;
an ammonium cation of formula III: 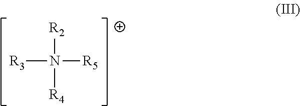
wherein R₂, R₃, R₄ and R₅ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or two of R₂, R₃, R₄ and R₅ are linked to form a heterocyclic group; and
a guanidinium cation of formula (IV): 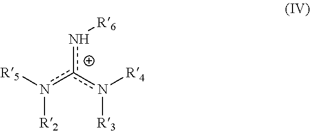
wherein R′₂, R′₃, R′₄, R′₅ and R′₆ are each independently selected from the group consisting of: H, alkyl, alkenyl, alkynyl, hydroxyalkyl, poly(hydroxy)alkyl, cycloalkyl, alkylaryl, arylalkyl, aminoaryl, aminoalkyl, aminoalkenyl, aminoalkynyl, arylalkylaminoalkyl and alkylaminoaryl; or wherein two of R′₂, R′₃, R′₄, R′₅ and R′₆ are linked to form a heterocyclic group;
and wherein said process comprises:
i. when R₁ⁿ⁺ is a metal cation:
i.a. contacting a solution of the compound of formula I with said metal cation;
i.b. contacting a solution of the compound of formula I with a first cation to form a salt of the compound of formula I and said first cation; and contacting said salt of the compound of formula I and said first cation with said metal cation; or
i.c. contacting a solution of the compound of formula I with the metal from which said metal cation is derived or an inorganic compound of said metal;
ii. when R₁ⁿ⁺ is an amino acid cation:
ii.a contacting a solution of the compound of formula I with said amino acid cation;
ii.b. contacting a solution of the compound of formula I with a first cation to form a salt of the compound of formula I and said first cation; and contacting said salt of the compound of formula I and said first cation with said amino acid cation; or
ii.c contacting a solution of the compound of formula I with the amino acid from which said amino acid cation is derived by protonation;
iii. when R₁ⁿ⁺ is an ammonium cation of formula III:
iii.a. contacting a solution of the compound of formula I with said ammonium cation of formula III;
iii.b contacting as solution of the compound of formula I with a first cation to form a salt of the compound of formula I and said first cation; and contacting said salt of the compound of formula I and said first cation with said ammonium cation of formula III; or
iii.c. and when R₅ is H, contacting a solution of the compound of formula I with the amine of formula V from which said ammonium cation of formula III is derived by protonation: 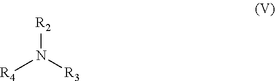
iv. when R₁ⁿ⁺ is a guanidinium cation of a guanidine derivative of formula IV:
iv.a. contacting the compound of formula I with said guanidinium cation of formula IV;
iv.b. contacting as solution of the compound of formula I with a first cation to form a salt of the compound of formula I and said first cation; and contacting said salt of the compound of formula I and said first cation with said guanidinium cation of formula IV; or
iv.c. contacting a solution of the compound of formula I with said guanidine derivative of formula IVb from which said guanidium cation of formula IV is derived by protonation: 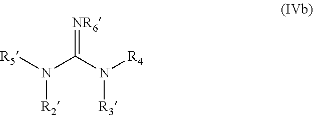
wherein n is a number selected from the group consisting of: 1, 2, 3 and 4.

13. A compound of formula I, or a pharmaceutical salt thereof of formula II, according to claim 1, for use as a medicament.

14. A compound of formula I, or a pharmaceutical salt thereof of formula II, according to claim 1, for use in the treatment or prevention of a disease responsive to PPARγ agonists.

15. A compound of formula I, or a pharmaceutical salt thereof of formula II for use, according to claim 14, wherein the disease responsive to PPARγ agonists is selected from the group consisting of: atherosclerosis, inflammatory bowel diseases, rheumatoid arthritis, liver fibrosis, nephropathy, psoriasis, skin wound healing, skin regeneration, pancreatitis, gastritis, neurodegenerative disorders, neuroinflammatory disorders, scleroderma, cancer, hypertension, obesity and type II diabetes.

16. A method for treating or preventing a disease responsive to PPARγ agonists comprising administering to a patient an effective amount of a compound of formula I or of a pharmaceutical salt thereof of formula II, according to claim 1.

17. The method of claim 16, wherein the disease responsive to PPARγ agonists is selected from the group consisting of: atherosclerosis, inflammatory bowel diseases, rheumatoid arthritis, liver fibrosis, nephropathy, psoriasis, skin wound healing, skin regeneration, pancreatitis, gastritis, neurodegenerative disorders, neuroinflammatory disorders, scleroderma, cancer, hypertension, obesity and type II diabetes.
